(19) United States
(12) Patent Application Publication
(10) Pub. No.: US 2026/0235602 A1
(43) Pub. Date: Aug. 13, 2026

(54) NANOBIOSENSOR AND PREPARATION METHOD THEREOF AND VIRUS ANTIBODY DETECTION METHOD USING SAME

(71) Applicant: National Defense Medical University, Taipei City (TW)

(72) Inventors: Szu-Cheng Kuo, Taipei City (TW); Yu-Lin Hsu, Taipei City (TW); Der-Jiang Chiao, Taipei City (TW)

(21) Appl. No.: 19/395,264

(22) Filed: Nov. 20, 2025

(30) Foreign Application Priority Data
Feb. 8, 2025 (TW) 114104764

(51) Int. Cl. G01N 33/569; C12N 7/00; G01N 33/543

(52) U.S. Cl. CPC G01N 33/56983; C12N 7/00; G01N 33/5434; C12N 2770/36122; C12N 2770/36123; G01N 2333/181; G01N 2469/20

(57) ABSTRACT

A nanobiosensor includes a virus-like particle and a luciferase. The virus-like particle is assembled by a structural protein of a virus. The luciferase forms a fusion protein with the structural protein of the virus and is displayed on the surface of the virus-like particle. The virus-like particle is capable of capturing specific antibodies against the virus, and the luciferase is analyzed through a luciferase immunoprecipitation system, which enables a rapid detection of the specific antibodies against the virus.

### CROSS-REFERENCE TO RELATED APPLICATION

This application claims priority to Taiwan Patent Application No. 114104764, filed on Feb. 8, 2025, the entire contents of which are incorporated herein by reference for all purposes.

### INCORPORATION BY REFERENCE

The Sequence Listing XML having the file name “08812PUS.xml”, a production date of Nov. 11, 2025, and a file size of 45,803 bytes is incorporated herein by reference for all purposes.

### FIELD OF THE INVENTION

The present disclosure relates to a nanobiosensor, and more particularly to a nanobiosensor used for detecting virus antibodies and a detection method thereof.

### BACKGROUND OF THE INVENTION

Mosquito-borne viruses are viruses that are transmitted by mosquitoes. The mosquito-borne viruses are numerous and widely distributed, and they spread diseases through mosquito bites. Urbanization, global warming and increasing international travel have also accelerated the spread of mosquito-borne diseases. For example, Chikungunya infections have posed a major threat to public health.

Chikungunya virus (CHIKV) belongs to the genus Alphavirus in taxonomy. Humans infected with the alphavirus may experience symptoms such as acute encephalitis, neurological diseases, acute and chronic musculoskeletal diseases and arthritis. For example, the infection with CHIKV causes Chikungunya fever, characterized by symptoms such as high fever, severe joint pain, headache, muscle pain, rash, and fatigue. The joint pain associated with CHIKV infection can be debilitating and long lasting from weeks to months or even years in some cases, influencing the quality of life and productivity of affected individuals. The treatment to CHIKV infection focuses on symptom relief and supportive care only since no specific antiviral treatment is available currently for CHIKV infection. Therefore, improved diagnostic tools for antibodies could mitigate CHIKV's spread and impact.

Numerous serological tests for detecting CHIKV-specific antibodies have been developed and commercialized. However, the existing detection methods are often labor-intensive and time-consuming. For example, the conventional ELISA antibody detection has complicated steps and takes two hours to complete the test, making it difficult to be applied to rapid antibody detection and point-of-care testing (POCT). Therefore, in order to effectively combat such diseases and distinguish CHIKV infection from other mosquito-borne virus infections, it is necessary to further develop reliable, sensitive, rapid and specific detection systems and methods to enhance diagnosis and epidemic prevention.

### SUMMARY OF THE INVENTION

An object of the present disclosure is to provide a novel nanobiosensor being an enzyme-functionalized virus-like particle (VLP) to efficiently and rapidly detect virus antibodies and overcome the drawbacks of the conventional antibody detection methods.

Another object of the present disclosure is to provide a virus antibody detection method, which uses the nanobiosensor developed in the present disclosure to bind with specific antibodies against a virus, and employs luciferase immunoprecipitation system (LIPS) for antibody assay, so as to achieve rapid detection of virus antibodies and further application in POCT.

According to an aspect of the present disclosure, there is provided a nanobiosensor including a virus-like particle and a luciferase. The virus-like particle is assembled by a structural protein of a virus. The luciferase forms a fusion protein with the structural protein of the virus and is displayed on the surface of the virus-like particle. The virus-like particle is capable of capturing specific antibodies against the virus, and the luciferase is analyzed through a luciferase immunoprecipitation system, which enables a rapid detection of the specific antibodies against the virus.

In an embodiment, the virus-like particle does not contain a capsid protein of the virus.

In an embodiment, the virus is a mosquito-borne virus.

In an embodiment, the virus is an alphavirus or a flavivirus.

In an embodiment, the alphavirus includes Chikungunya virus, Venezuelan equine encephalitis virus, Eastern equine encephalitis virus, Western equine encephalitis virus, Mayaro virus, Ross River virus, Barmah forest virus, O'nyong-nyong virus, and Sindbis virus.

In an embodiment, the flavivirus includes Dengue virus, Japanese encephalitis virus, Zika virus, West Nile virus, Yellow fever virus, and Hepatitis C virus.

In an embodiment, the luciferase is Lucia luciferase.

In an embodiment, the nanobiosensor includes proteins of SEQ ID NO. 3 and SEQ ID NO. 4.

In an embodiment, the nanobiosensor includes proteins of SEQ ID NO. 7 and SEQ ID NO. 8.

In an embodiment, the nanobiosensor includes proteins of SEQ ID NO. 11 and SEQ ID NO. 12.

In an embodiment, the nanobiosensor includes a protein of SEQ ID NO. 15.

In an embodiment, the nanobiosensor includes proteins of SEQ ID NO. 18 and SEQ ID NO. 19.

In an embodiment, the nanobiosensor further includes an additional antigen, which forms a fusion protein with the luciferase and is displayed on the surface of the virus-like particle.

According to another aspect of the present disclosure, there is provided a preparation method of a nanobiosensor, characterized by inserting a luciferase gene into a structural protein gene of a virus, and using a baculovirus/mosquito system to express, assemble and secrete a virus-like particle, wherein an expressed luciferase protein and an expressed structural protein of the virus form a fusion protein displayed on a surface of the virus-like particle.

According to a further aspect of the present disclosure, there is provided a detection method of virus antibodies, including steps of: (a) providing a nanobiosensor and magnetic beads for capturing human IgG or IgM, wherein the nanobiosensor includes a virus-like particle assembled by a structural protein of a virus and a luciferase forming a fusion protein with the structural protein of the virus and displayed on the surface of the virus-like particle; (b) mixing the nanobiosensor, the magnetic beads and a sample; (c) washing the magnetic beads to remove non-specific bindings; and (d) adding a substrate of the luciferase and detecting a light signal.

In an embodiment, the magnetic beads are protein G-conjugated magnetic beads or anti-human IgM antibody conjugated magnetic beads.

In an embodiment, the steps (b) and (c) further include steps of: (b1) mixing the magnetic beads and the sample; (c1) washing the magnetic beads to remove non-specific bindings; (b2) adding the nanobiosensor for further mixing; and (c2) washing the magnetic beads to remove non-specific bindings.

In an embodiment, in the step (d), the light signal is detected by a smartphone or a portable luminometer.

In an embodiment, in the step (b), the sample comprises serum, blood, saliva, and monoclonal antibody.

The above objects and advantages of the present disclosure will become more readily apparent to those ordinarily skilled in the art after reviewing the following detailed description and accompanying drawings, in which:

### BRIEF DESCRIPTION OF THE DRAWINGS

FIG. 1 shows the preparation method of the CHIK VLP-Lucia;

FIG. 2 shows the immunofluorescence results of the transduced mosquito cells;

FIG. 3 shows the western blot results using the E2 antibodies, the E1 antibodies and the Lucia antibodies for detection;

FIG. 4 shows the luciferase assay results;

FIG. 5 shows the particle analysis results of the CHIK VLP-Lucia;

FIG. 6 shows the protein structure modeling of the Lucia-E2 and E1 protein complex;

FIG. 7 shows a schematic view of the LIPS assay using the CHIK VLP-Lucia;

FIG. 8 shows the LIPS assay results using the CHIK VLP-Lucia;

FIG. 9 shows the SEM analysis of the CHIK VLP-Lucia LIPS assay;

FIG. 10 shows the condition analysis of the CHIK VLP-Lucia LIPS assay;

FIG. 11 shows the sensitivity analysis of the CHIK VLP-Lucia LIPS assay;

FIG. 12 shows the LIPS assay results to the 52 samples by the CHIK VLP-Lucia provided in the present disclosure;

FIG. 13 shows the LIPS assay results to the simulated whole blood samples by the CHIK VLP-Lucia provided in the present disclosure;

FIG. 14 shows the LIPS assay results to the simulated saliva samples by the CHIK VLP-Lucia provided in the present disclosure;

FIG. 15 shows a schematic view of the CHIK VLP-Lucia LIPS assay applied to POCT;

FIG. 16 shows another schematic view of the CHIK VLP-Lucia LIPS assay applied to POCT;

FIG. 17 shows the smartphone detection performance of the CHIK VLP-Lucia LIPS assay;

FIG. 18 shows the preparation method of the VEE VLP-Lucia;

FIG. 19 shows the preparation method of the MAY VLP-Lucia;

FIG. 20 shows the preparation method of the DEN VLP-Lucia;

FIG. 21 shows the LIPS assay results to the 20 samples by the DEN VLP-Lucia provided in the present disclosure;

FIG. 22 shows the preparation method of the CHIK VLP-DIII-Lucia; and

FIG. 23 shows the LIPS assay results to the 26 samples by the CHIK VLP-DIII-Lucia provided in the present disclosure.

### DETAILED DESCRIPTION OF THE PREFERRED EMBODIMENT

The invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

Several nanomaterials, including metal nanoparticles and protein nanoparticles, have been employed to enhance detection signals in biosensing. These approaches improve detection sensitivity significantly compared to conventional methods, positioning nanomaterials as promising candidates for ultrasensitive diagnostic probes. In order to overcome the drawbacks of the conventional antibody detection methods that are labor-intensive and time-consuming, the present disclosure is committed to developing a new antibody diagnostic tool and attempting to prepare a nanobiosensor using the protein nanoparticles, so as to be applied in rapid detection of virus antibodies. However, due to inherent surface area limitations, such materials often face challenges in designing carriers that can display multivalent enzymes. Additionally, uniformly sized nanostructures are difficult to obtain and may become unstable after surface biological functionalization, leading to low-yielding issues. Thus, designing functional nanoprobes for highly sensitive nanobiosensors remains a challenge.

The virus-like particle (VLP) is a particle assembled from structural protein(s) of a virus. The VLP mimics the original three-dimensional structure of the virus but do not contain viral nucleic acids, so it is non-infectious virus particle and cannot re-infect or self-replicate. These non-infectious protein particles produced by genetic recombination are well developed and applied in vaccine development to improve the safety of viral vaccine preparation and use. The VLP, biomimetic protein-based nanoparticle, has the structural protein(s) of the virus and thus can be used in ELISA for antibody detection, but the detection time still takes 2 hours. If the VLP can be further enzyme-functionalized, it will be able to offer necessary molecular recognition functions for antibody detection while preserving physicochemical properties required for effective translation of nanomaterials, such as high surface-area-to-volume ratio and multivalent effects, thereby improving detection efficiency. Consequently, the enzyme-functionalized VLP will be able to serve as the nanoprobe to advance biomedical applications including biomolecular sensing.

Therefore, the present disclosure further develops the enzyme-functionalized VLP to express the luciferase protein on the surface of the VLP, so as to employ the luciferase immunoprecipitation system (LIPS) for antibody assay. The following will take Chikungunya virus (CHIKV) as an example to illustrate the nanobiosensor preparation of the present disclosure.

To express the luciferase protein on the surface of the VLP for antibody assay, it is necessary to retain the original enzyme function of the luciferase protein, not to destroy the antigenicity of the viral protein, and to maintain the structural stability of the VLP. Accordingly, the preparation method of the nanobiosensor in the present disclosure inserts the luciferase gene into the structural protein gene of the CHIKV, and employs the baculovirus/mosquito (BacMos) system to express, assemble and secrete the VLP. Particularly, the luciferase protein and the structural protein of the CHIKV form a fusion protein, which is displayed on the surface of the VLP.

The luciferase may be Firefly luciferase (F-Luc), Cypridina luciferase (C-Luc), Renilla luciferase (R-Luc), Luciola italica luciferase (Red-Luc), Gaussia luciferase (Gluc), nano-luciferase (N-Luc), and Lucia luciferase (Lucia). Different luciferases have different substrates and emit different wavelengths of light. In an embodiment, the luciferase employed in the present disclosure is Lucia, which can catalyze the oxidation reaction of the substrate coelenterazine, accompanied by the release of light, so it can be detected through light signals.

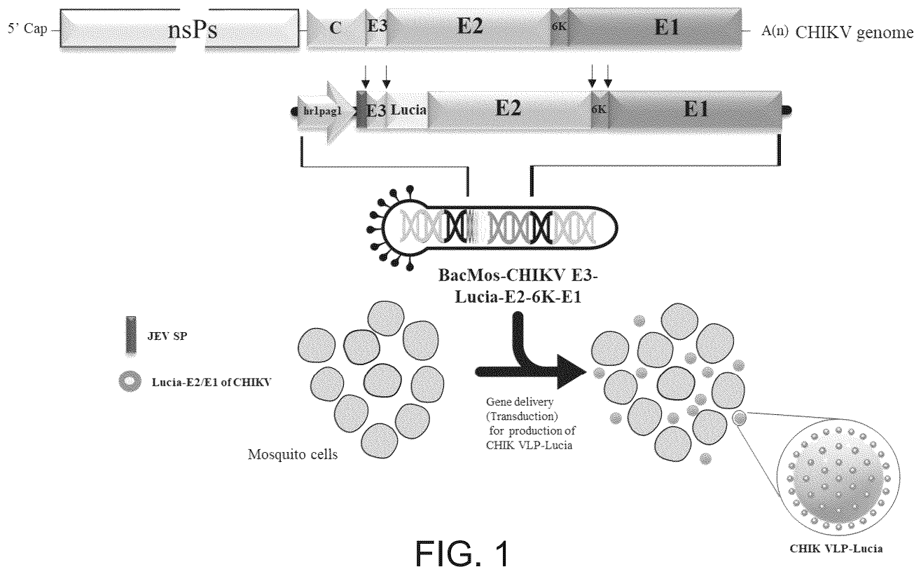

According to the above, the present disclosure provides the approach to express, assemble and secrete the VLP displaying the fusion protein of the CHIKV structural protein and the luciferase protein (hereinafter referred as CHIK VLP-Lucia) through BacMos system. FIG. 1 shows the preparation method of the CHIK VLP-Lucia. There are genes encoding the structural proteins and the non-structural proteins (nsPs) in the CHIKV genome. The structural proteins include the capsid (C) protein and the envelope proteins of 6K, E1, E2 and E3. The genetic recombination design is to insert the Lucia gene between the E2 gene and the E3 gene of the CHIKV (i.e. in-frame insertion at junction of E3-E2), delete the C gene and add a mosquito promoter hr1pag1 and a signal peptide JEV SP to form a gene cassette of hr1pag1-JEV SP-CHIKV E3-Lucia-E2-6K-E1. The gene cassette was recombined with the baculovirus gene, and then the recombinant baculovirus (represented by BacMos-CHIKV E3-Lucia-E2-6K-E1) was transduced into mosquito cells for gene delivery, thereby inducing the expression of the CHIK VLP-Lucia. During the expression processing, the E3 protein and the 6K protein were cleaved (protease cleavage sites indicated by the arrows), resulting in the formation and secretion of the CHIK VLP-Lucia having the fusion protein of E1/E2 and Lucia.

Since the capsid protein of the alphavirus is responsible for host transcriptional repression and subsequent cytopathic effects, removal of the capsid protein can reduce cell damage. Therefore, the genetic recombination design of the present disclosure also deletes the C gene simultaneously, so that the expressed VLP does not contain the capsid protein of the CHIKV.

In an embodiment, the recombinant sequence of JEV SP-CHIKV E3-Lucia-E2-6K-E1 can be created by first synthesizing the individual JEV SP-CHIKV E3 segment, Lucia segment, and CHIKV E2-6K-E1 segment, and then combining the three segments to form the complete recombinant sequence of JEV SP-CHIKV E3-Lucia-E2-6K-E1 (SEQ ID NO. 1). In the recombinant sequence, the base positions 29 to 3655 are encoding sequence, which encodes 1209 amino acids (SEQ ID NO. 2). In addition, during the protein expression processing, the E3 protein and the 6K protein were cleaved, so the secreted product CHIK VLP-Lucia included the Lucia-CHIKV E2 protein (SEQ ID NO. 3) and the CHIKV E1 protein (SEQ ID NO. 4).

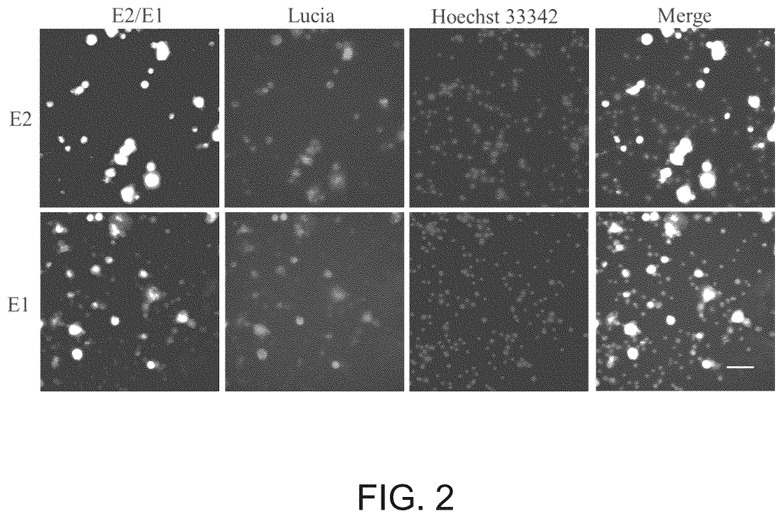

In an embodiment, the mosquito cells used in the present disclosure were C6/36 mosquito cells from Aedes albopictus. After transduction by the recombinant baculovirus for three days, the transduced cells were stained using either E2 antibodies (anti-CHIKV E2 antibodies) or E1 antibodies (anti-CHIKV E1 antibodies), Lucia antibodies (rabbit anti-Gluc serum), and nucleic acid dyes (Hoechst 33342), and then observed under a fluorescence microscope. FIG. 2 shows the immunofluorescence results of the transduced mosquito cells, wherein the upper panels show the cells co-stained with the E2 antibodies, the Lucia antibodies and the nucleic acid dyes, the lower panels show the cells co-stained with the E1 antibodies, the Lucia antibodies and the nucleic acid dyes, and the rightmost panels show the merged images. According to the staining results, the E2 protein, the E1 protein and the Lucia protein were indeed co-expressed in the transduced mosquito cells.

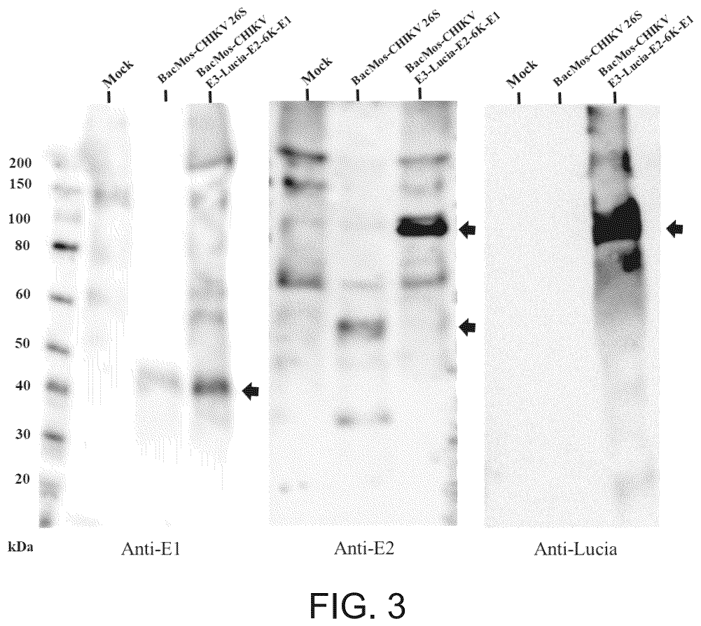

The culture supernatants from the transduced mosquito cells were subjected to the western blot to analyze the proteins secreted from the transduced mosquito cells. The compared cells included the cells transduced by BacMos-CHIKV E3-Lucia-E2-6K-E1 (with Lucia insertion), the cells transduced by BacMos-CHIKV 26S (without Lucia insertion), and the untreated cells (Mock). The cells transduced by BacMos-CHIKV 26S were supposed to express the CHIK VLP, and the cells transduced by BacMos-CHIKV E3-Lucia-E2-6K-E1 were supposed to express the CHIK VLP-Lucia. FIG. 3 shows the western blot results using the E2 antibodies, the E1 antibodies and the Lucia antibodies for detection. According to the results, E1 proteins of comparable size were detected in both transduced cells. Further, an E2 protein band migrating to the 90 kDa (Lucia-E2) gel position was observed from the BacMos-CHIKV E3-Lucia-E2-6K-E1-transduced cells, compared to the 55 kDa (E2) gel position from the BacMos-CHIKV 26S-transduced cells. Additionally, a corresponding protein band at the 90 kDa (Lucia-E2) gel position was detected using the Lucia antibody from BacMos-CHIKV E3-Lucia-E2-6K-E1-transduced cells only. Consequently, the BacMos-CHIKV E3-Lucia-E2-6K-E1-transduced cells designed in the present disclosure indeed successfully secrete the E1 proteins, the E2 proteins and the Lucia proteins, and the Lucia proteins were fused with the E2 proteins.

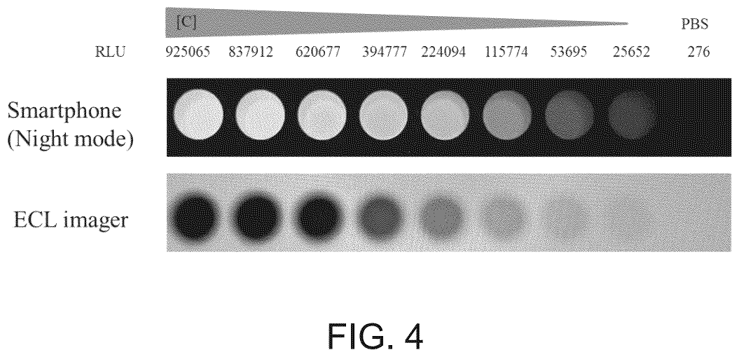

Next, the fusion proteins (Lucia-E2) were assayed for luciferase activity. A 2-fold serial dilution of the culture supernatant from the BacMos-CHIKV E3-Lucia-E2-6K-E1-transduced mosquito cells was subjected for detection of luciferase activity to obtain relative light units (RLU) using a luminometer, or to capture images using a smartphone or an enhanced chemiluminescence (ECL) imager. FIG. 4 shows the luciferase assay results. It is clear from FIG. 4 that the luciferase activity was detected from the Lucia-E2 fusion proteins secreted by the transduced mosquito cells. The luciferase activity can be easily detected by the smartphone, demonstrating potential for point-of-care testing (POCT).

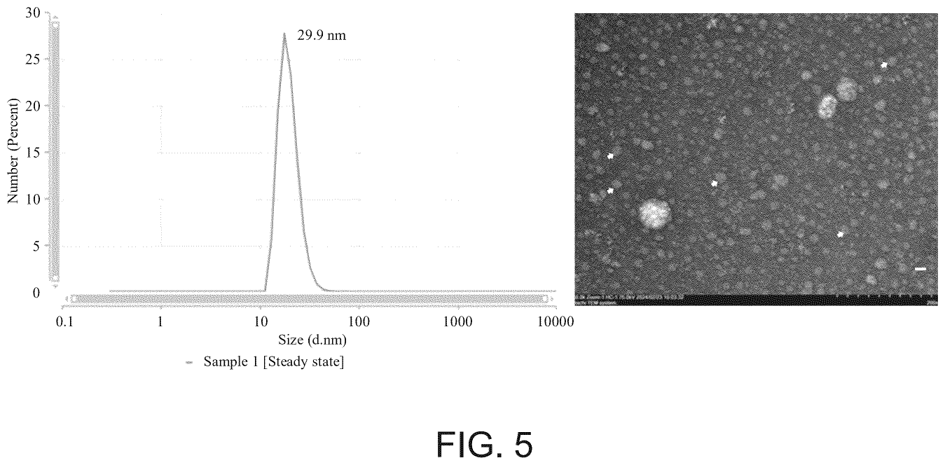

To confirm the secretion of the Lucia-E2 and E1 proteins in VLP format, the present disclosure used the transmission electron microscopy (TEM) to observe the CHIK VLP-Lucia, and used the dynamic light scattering (DLS) to analyze the size distribution of the CHIK VLP-Lucia. FIG. 5 shows the particle analysis results of the CHIK VLP-Lucia, wherein the left panel shows the DLS analysis, and the right panel shows the TEM image where the VLPs are indicated by white arrows. The TEM image revealed the presence of the CHIK VLP-Lucia as spherical particles with an average diameter of 30 nm, and these measurements were confirmed by DLS analysis.

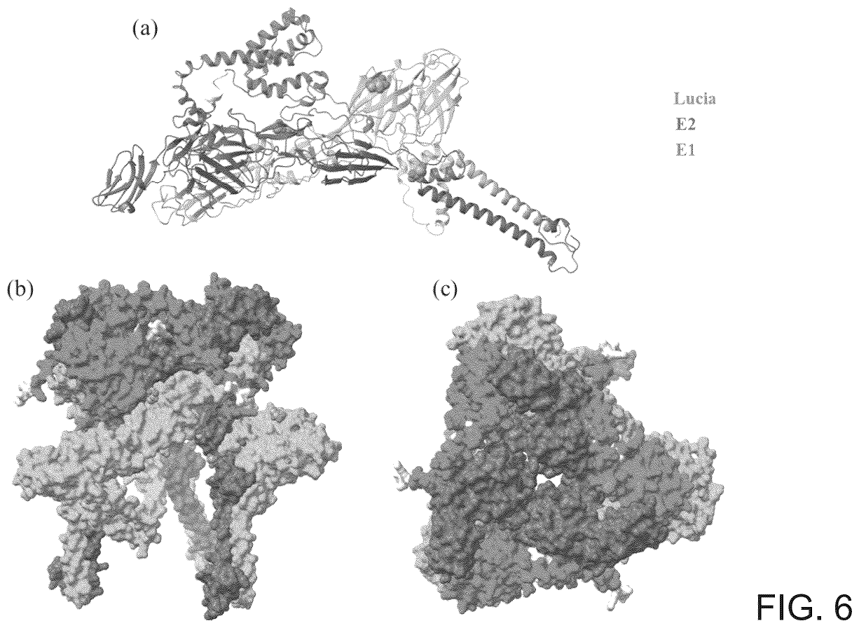

Alphafold3 was further used to model the chimeric Lucia-E2 and E1 protein complex. FIG. 6 shows the protein structure modeling of the Lucia-E2 and E1 protein complex. The E1 proteins, the E2 proteins and the Lucia proteins are shown in different gray-scale colors, respectively. The subfigure (a) shows the ribbon diagram of E1/Lucia-E2 heterodimer, and the subfigures (b) and (c) show surface views of one viral spike from the side and the top, respectively. The protein structure modeling in FIG. 6 revealed that the quaternary structure of Lucia-E2 and E1 was similar to the published crystal structure of the E2/E1 dimer of CHIKV, indicating that the 3D structure of E2/E1 did not noticeably change upon Lucia fusion with the N-terminal tail of the E2 glycoprotein. This suggests that the genetic fusion of CHIKV E2 with Lucia does not alter the antigenicity or structural integrity of the CHIKV E2/E1 dimer. Thus, the VLP-Lucia serves as a native bait for capturing target-specific antibodies and a sensitive probe. The luminescent signals directly reported from the VLP-Lucia eliminate the need for conjugated secondary antibodies, which avoids cross-reactions and high background noise due to non-specific binding.

From the above, it is confirmed that the BacMos system is able to produce the CHIK VLP-Lucia, i.e. the biomimetic Chikungunya virus-like particles labeled with the Lucia protein. The following will further verify whether the CHIK VLP-Lucia can be used as a nanobiosensor for antibody detection.

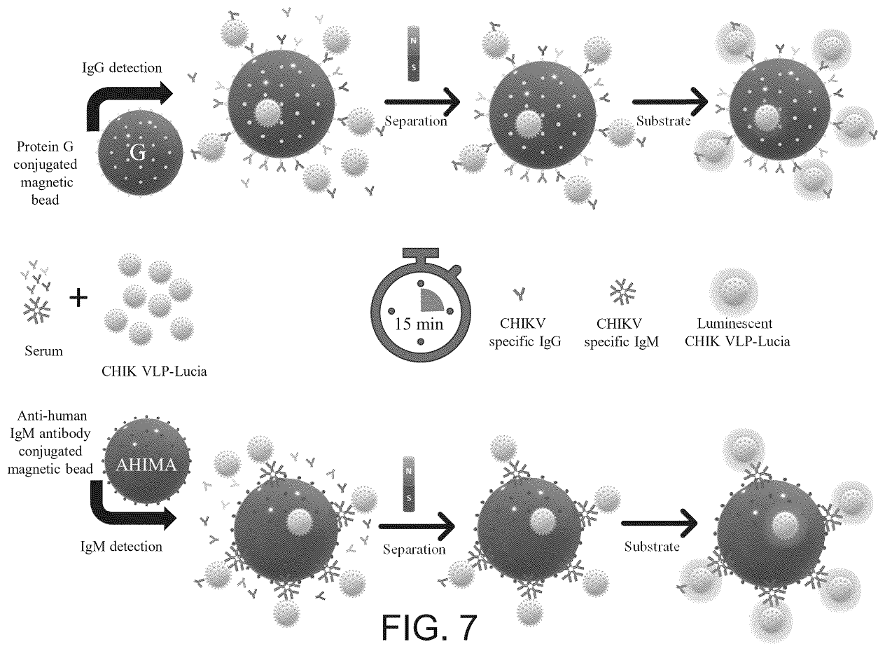

FIG. 7 shows a schematic view of the LIPS assay using the CHIK VLP-Lucia. For IgG detection, firstly, the CHIK VLP-Lucia, the patient serum samples, and the protein G-conjugated magnetic beads were mixed for 10 minutes. At the moment, the CHIK VLP-Lucia, the CHIKV-specific IgG in the patient serum samples, and the protein G on the magnetic beads formed a sandwich immune complex. After the magnetic beads were washed using a magnetic cylinder, the substrate of Lucia was added, and then the light signal was further detected and quantified. For IgM detection, firstly, the CHIK VLP-Lucia, the patient serum samples, and the anti-human IgM antibody conjugated magnetic beads (AHIMA) were mixed for 10 minutes. At the moment, the CHIK VLP-Lucia, the CHIKV-specific IgM in the patient serum samples, and the antibodies on the magnetic beads formed a sandwich immune complex. After the magnetic beads were washed using a magnetic cylinder, the substrate of Lucia was added, and then the light signal was further detected and quantified.

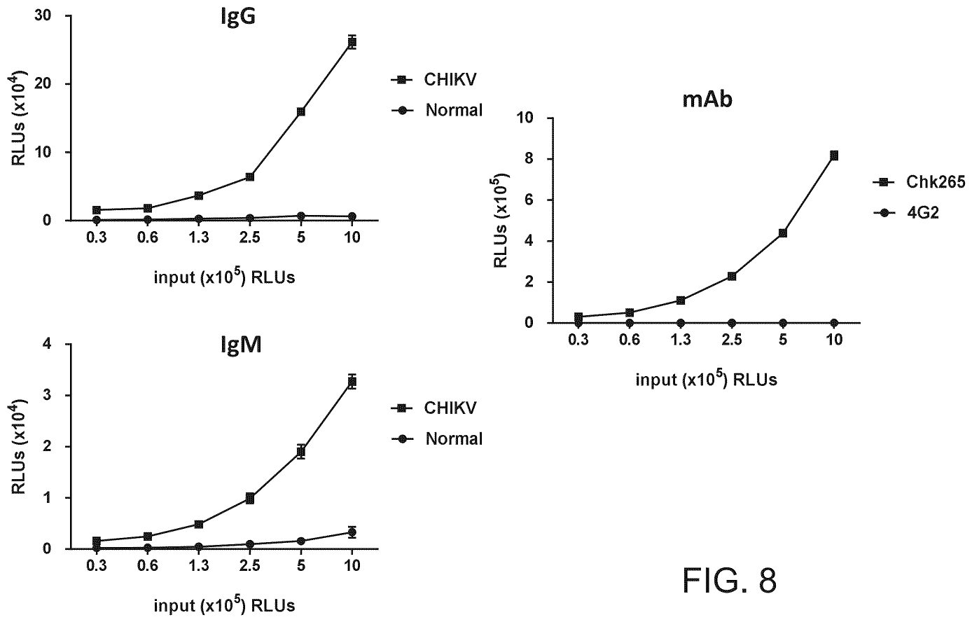

FIG. 8 shows the LIPS assay results using the CHIK VLP-Lucia. In addition to detect specific IgG and IgM in patient serums (CHIKV) and normal serums (Normal), a sample containing CHIKV monoclonal antibodies (mAb) Chk265 was used as a positive control and a sample containing anti-Flavivirus envelope protein mAb 4G2 was used as a negative control. In the analysis graphs, the horizontal axis is the input of the CHIK VLP-Lucia, expressed as input RLUs, and the vertical axis is the light signal measured by LIPS, expressed as RLUs. FIG. 8 clearly demonstrates that the CHIK VLP-Lucia LIPS assay exhibited dose-dependent signal outputs for IgG, IgM and mAb upon increasing the amount of input CHIK VLP-Lucia in positive antibody samples (Chk265 and CHIKV patient serum), while showing no reactivity to negative controls (4G2 and normal serum). Further, significant high values of RLU reached when 5×10⁵ input RLUs of the CHIK VLP-Lucia were used.

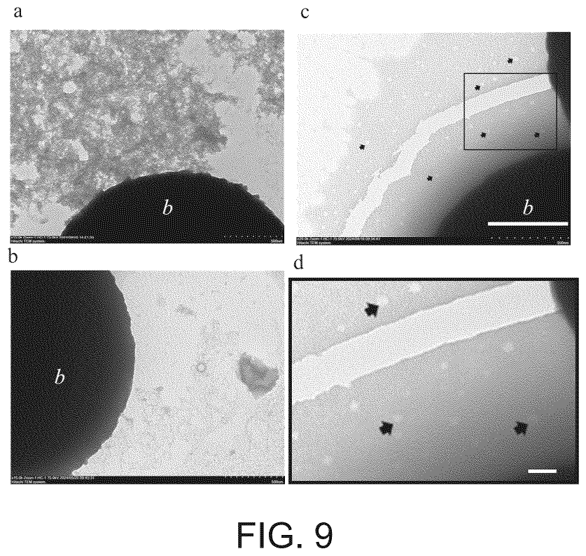

The CHIK VLP-Lucia LIPS assay was further characterized using the scanning electron microscope (SEM). FIG. 9 shows the SEM analysis of the CHIK VLP-Lucia LIPS assay. The subfigure a was from the mixed sample of the protein G-conjugated magnetic beads and the CHIK VLP-Lucia, the subfigure b was from the mixed sample of the protein G-conjugated magnetic beads and the CHIK VLP-Lucia with the negative mAb 4G2, the subfigure c was from the mixed sample of the protein G-conjugated magnetic beads and the CHIK VLP-Lucia with the positive mAb Chk265, and subfigure d was an enlarged view of the box in the subfigure c (scale bar is 50 nm). In the figures, “b” indicated the magnetic beads, and the CHIK VLP-Lucia was indicated by black arrows. From the subfigures c and d, abundant CHIK VLP-Lucia particles were captured on the protein G-conjugated magnetic beads in the presence of the positive mAb Chk265. While in the two negative control experiments shown in the subfigures a and b, no CHIK VLP-Lucia particles were captured on the protein G-conjugated magnetic beads. This confirms that the capture of the CHIK VLP-Lucia in the LIPS assay is specific to antibodies against CHIKV, rather than resulting from non-specific adsorptions.

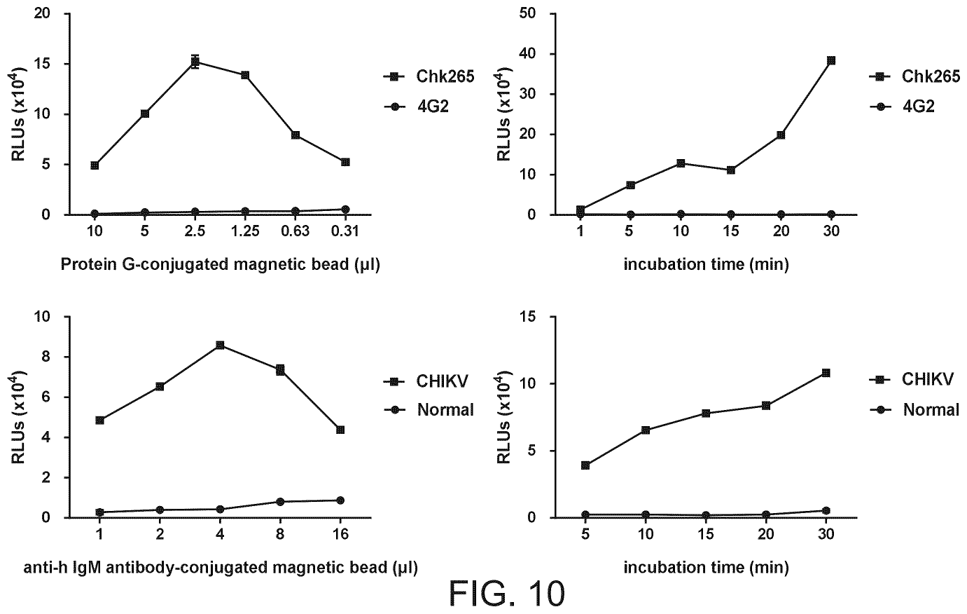

The present disclosure further optimized the test conditions of the CHIK VLP-Lucia LIPS assay, wherein two conditions were considered crucial for its performance: the amount of the magnetic beads and the incubation time. FIG. 10 shows the condition analysis of the CHIK VLP-Lucia LIPS assay. Analysis of the signal-to-noise (S/N) ratios of the positive mAb (Chk265) and the negative mAb (4G2) was used to optimize the test conditions. The assay was fixed at 5×10⁵ RLUs for CHIK VLP-Lucia input, 10 ng for mAb, and 1/100 dilution for human serum per reaction. According to the analysis results, the peak RLU with a high S/N ratio reached its maximal value when 2.5 μl of the protein G-conjugated magnetic beads was used. Additionally, a robust signal output in the presence of Chk265 was detected after 5 minutes of incubation, indicating that the CHIK VLP-Lucia LIPS assay has a fast kinetics. The signal output increased with longer incubation times, with the RLU plateau in the presence of Chk265 being reached at 10 minutes. Altogether, 5×10⁵ RLUs of the CHIK VLP-Lucia, 2.5 μl of the protein G-conjugated magnetic beads, and 10 minutes for the incubation time were selected as the optimal conditions for the CHIK VLP-Lucia LIPS assay for IgG detection. For IgM detection, the optimal conditions were also determined to be 5×10⁵ RLUs of the CHIK VLP-Lucia, 4 μl of anti-human IgM antibody conjugated magnetic beads, and 10 minutes for the incubation time.

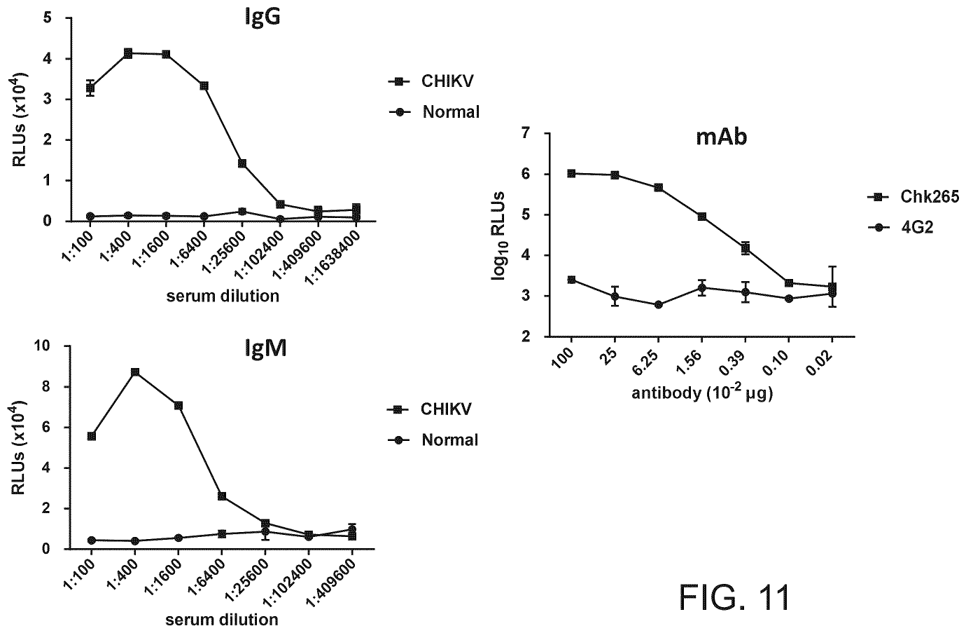

FIG. 11 shows the sensitivity analysis of the CHIK VLP-Lucia LIPS assay, and the assay was performed under the above-mentioned optimal conditions. According to the analysis results, 1 ng of the Chk265 mAb was sufficient to produce positive results for mAb detection. For IgG detection, the assay achieved maximum signal output with patient serum diluted 400 to 1,600 times, and positive IgG results were detectable at serum dilutions up to 25,600 times. While for IgM detection, the assay produced maximum signal output with patient serum diluted 400 times, and positive IgM results were detectable at serum dilutions up to 6,400 times.

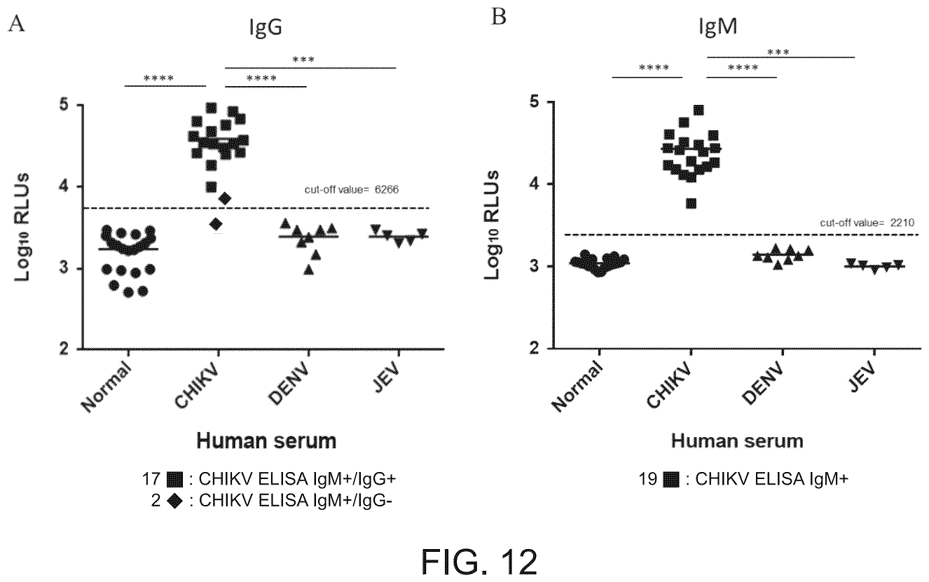

Multiple clinical samples were further used to analyze the detection efficacy of the CHIK VLP-Lucia LIPS assay. 52 laboratory-confirmed serum samples were tested. These samples included 19 from CHIKV-infected individuals (17 positives for both IgM and IgG by capture ELISA, and 2 positives for IgM but negative for IgG), 8 from DENV-infected individuals, 5 from JEV-infected individuals, and 20 from normal individuals. FIG. 12 shows the LIPS assay results to the 52 samples by the CHIK VLP-Lucia provided in the present disclosure. The test conditions were 5×10⁵ RLUs of the CHIK VLP-Lucia, 2.5 μl of the protein G-conjugated magnetic beads, 4 μl of anti-human IgM antibody conjugated magnetic beads, and 10 minutes for the incubation time. The serum was diluted 1,000 times for IgG detection and diluted 400 times for IgM detection. According to the results of FIG. 12, the CHIK VLP-Lucia LIPS assay effectively detected CHIKV-specific IgG and IgM. For IgG detection, all 17 capture ELISA IgG+ CHIKV control sera and 1 capture ELISA IgG− CHIKV control serum were classified as IgG positive by the CHIK VLP-Lucia LIPS assay. Conversely, all 34 non-CHIKV-related control sera and the 1 capture ELISA IgG− CHIKV control serum were classified as IgG negative. For IgM detection, all 19 capture ELISA IgM+ CHIKV control sera were classified as IgM positive by the CHIK VLP-Lucia LIPS assay, while all 33 non-CHIKV-related control sera were classified as IgM negative. Overall, the CHIK VLP-Lucia LIPS assay demonstrated a more sensitive detection than ELISA, achieving rapid and accurate results.

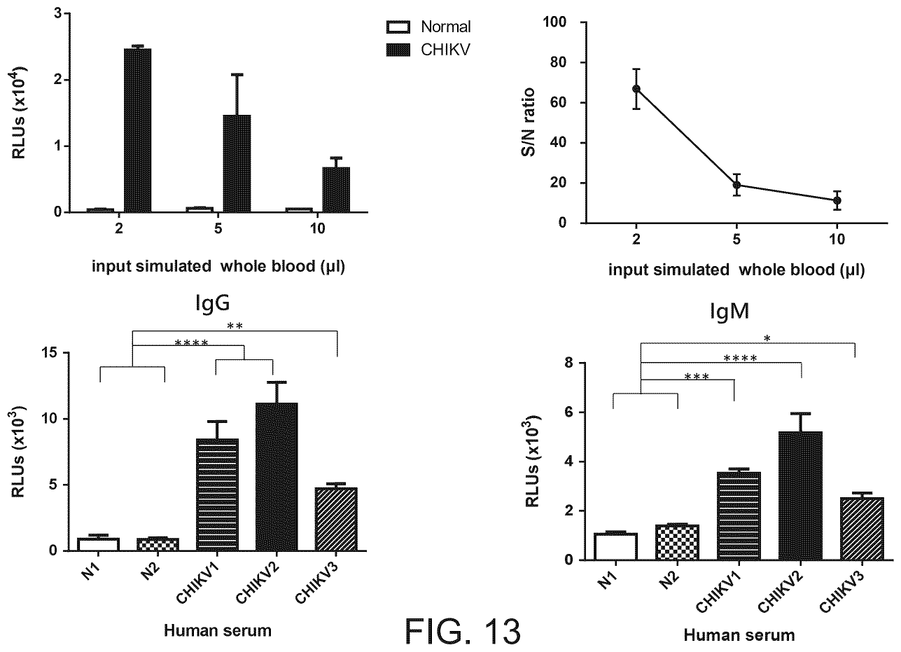

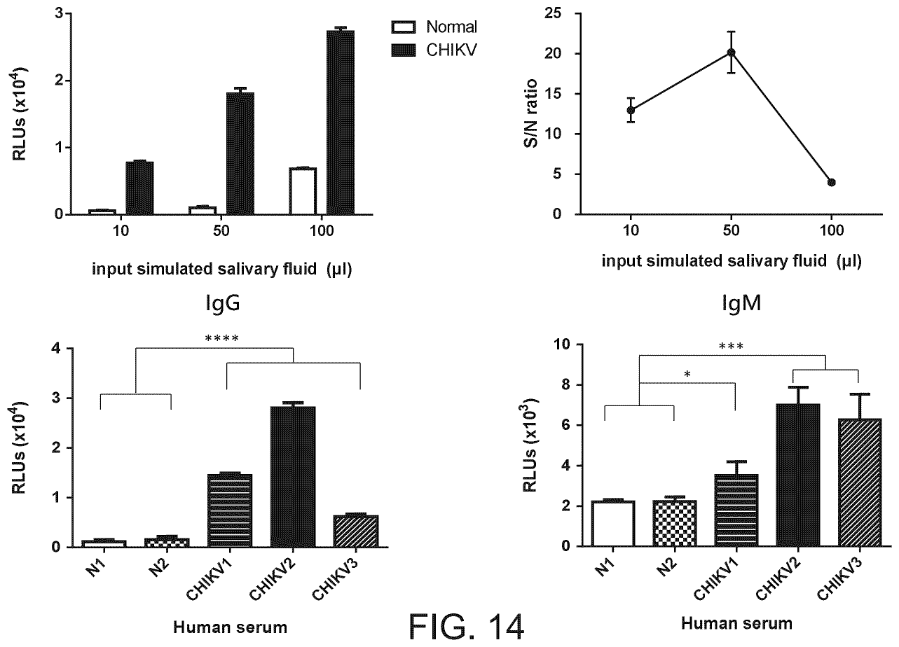

To develop the applications of the CHIK VLP-Lucia LIPS assay in POCT, the present disclosure tested the CHIK VLP-Lucia LIPS assay with simulated whole blood and saliva samples to assess the potential for on-site sampling and ensure accessibility and safety. Normal human whole blood or artificial saliva was spiked with serum samples from CHIKV patients or normal individuals to mimic their complexity. The CHIK VLP-Lucia LIPS assay was then optimized for these spiked samples. FIG. 13 shows the LIPS assay results to the simulated whole blood samples by the CHIK VLP-Lucia provided in the present disclosure, and FIG. 14 shows the LIPS assay results to the simulated saliva samples by the CHIK VLP-Lucia provided in the present disclosure.

First, the present disclosure evaluated the effect of whole blood sample volume on the CHIK VLP-Lucia LIPS assay. 2 μl, 5 μl and 10 μl of the whole blood samples were spiked with 1 μl of serum samples from either CHIKV patients or normal individuals. As shown in the upper panels of FIG. 13, the S/N ratios for 2 μl, 5 μl and 10 μl of the whole blood samples were 66.9, 19.0 and 11.2, respectively. The results indicated 2 μl of the whole blood sample spiked with 1 μl of the serum sample provided the optimal S/N ratio, and thus it was selected as the optimal condition per reaction for the simulated whole blood samples. Afterwards, one serum sample each from low, medium, and high antibody titer groups among CHIKV patients were selected as positive controls (n=3) and two normal sera were randomly selected as negative controls (n=2) to prepare simulated whole blood samples for the CHIK VLP-Lucia LIPS assay. As shown in the lower panels of FIG. 13, the CHIK VLP-Lucia LIPS assay accurately distinguished positive from negative samples in terms of IgG or IgM in the simulated whole blood samples.

Next, the present disclosure assessed the impact of saliva input volume on the CHIK VLP-Lucia LIPS assay. Artificial saliva spiked with human serum at a 1:1000 ratio was used to mimic physiological antibody concentrations. Input volumes of 10 μl, 50 μl and 100 μl of spiked saliva samples were tested. As shown in the upper panels of FIG. 14, the S/N ratios for 10 μl, 50 μl, and 100 μl of the input saliva were 13.0, 20.2 and 4.0, respectively, indicating 50 μl of the input saliva per reaction achieved the optimal S/N ratio. While 100 μl of the input saliva increased the RLU signal in patient samples, it also raised the background in normal samples. Therefore, 50 μl of the input saliva per reaction was selected as the optimal condition for the assay in simulated saliva samples. Afterwards, one serum sample each from low, medium, and high antibody titer groups among CHIKV patients were selected as positive controls (n=3) and two normal sera were randomly selected as negative controls (n=2) to prepare simulated saliva samples for the CHIK VLP-Lucia LIPS assay. As shown in the lower panels of FIG. 14, the CHIK VLP-Lucia LIPS assay also accurately distinguished positive from negative samples in terms of IgG or IgM in the simulated saliva samples.

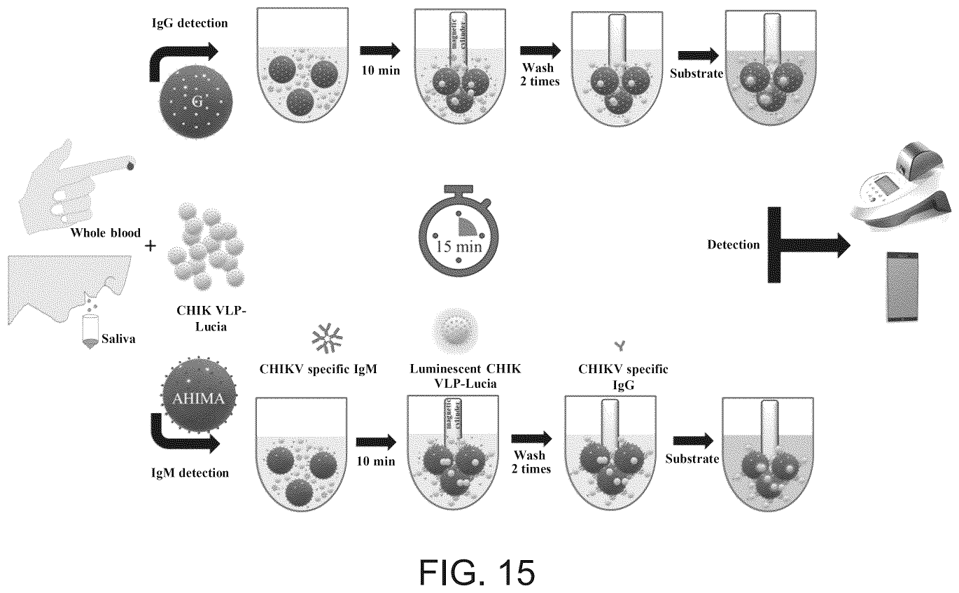

The above results demonstrate that the CHIK VLP-Lucia LIPS assay of the present disclosure has excellent ability to rapidly detect CHIKV antibodies in simulated whole blood or saliva, indicating the CHIK VLP-Lucia LIPS assay of the present disclosure has exceptional potential for the rapid on-site detection of CHIKV antibodies and can be further applied to POCT. FIG. 15 shows a schematic view of the CHIK VLP-Lucia LIPS assay applied to POCT. First, a sample, which may be a finger-prick whole blood sample (2 μl) or a saliva sample (50 μl), is collected at the point of care. Subsequently, the sample, the CHIK VLP-Lucia, and the magnetic beads capable of capturing IgG or IgM (e.g. the protein G-conjugated magnetic beads or the anti-human IgM antibody conjugated magnetic beads) are mixed for 10 minutes, and then the magnetic beads are washed twice using a magnetic cylinder to remove non-specific binding molecules. The washed magnetic beads are then placed into a tube with substrate for Lucia, and the light signals are measured using a portable luminometer or a smartphone to realize on-site rapid detection of antibodies.

In this testing platform, only a small amount of blood collected from simple finger prick is required for testing, which minimizes invasiveness compared to conventional blood collection methods, and also enhances patient compliance. The saliva, as a non-invasive alternative sample, offers the convenience of self-collection and home-based testing, supporting widespread testing. Furthermore, this testing platform does not require prior sample pretreatment and can obtain test results within 15 minutes. In addition, this testing platform can utilize small analytical devices to detect light signals, even use a smartphone to distinguish positive and negative samples, and exhibit dose-dependent signals. Therefore, the present disclosure develops a POCT platform using the CHIK VLP-Lucia LIPS assay to detect CHIKV antibodies on-site in non-invasive or low-invasive manners and obtain a rapid and accurate diagnosis. Accordingly, doctors can use the instant results obtained by the POCT to provide corresponding diagnosis or treatment immediately, and thus achieve epidemic control.

Since the light signal generated by the luciferase can be easily detected and has a highly linear light output, the antibodies can be detected without serum dilution, spanning a detection range often over seven orders of magnitude. Thus, it becomes a detection tool with high sensitivity, high linear dynamic range and high-throughput detection feasibility. In addition, most neutralizing antibodies recognize conformational epitopes, which ELISAs or other solid-phase assays poorly detect. The solution-phase LIPS assay utilized in the present disclosure efficiently detects these epitopes and, owing to the fast reaction kinetics of solution-phase assays, is ideal for developing quantitative POCT. Moreover, compared to the ELISA test which takes two hours, the CHIK VLP-Lucia LIPS assay of the present disclosure can obtain detection results quickly within 15 minutes, greatly improving the detection efficiency, and can be used as an excellent diagnostic tool.

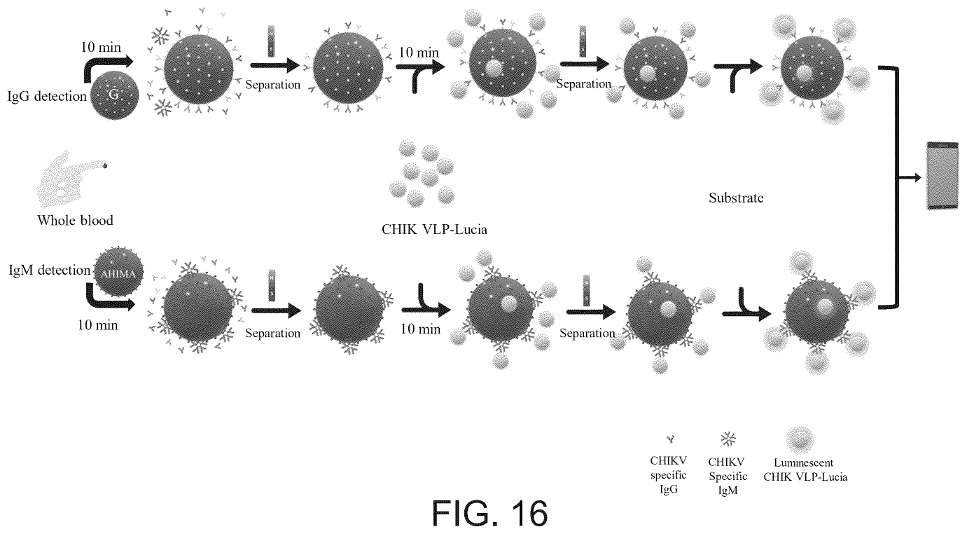

On the other hand, the present disclosure also provides an improved CHIK VLP-Lucia LIPS assay to enhance the light signal emitted from the reaction of the luciferase and the substrate, so that it can be directly detected by the smartphone. FIG. 16 shows another schematic view of the CHIK VLP-Lucia LIPS assay applied to POCT. In this improved method, the whole blood sample or the saliva sample and the magnetic beads capable of capturing IgG or IgM (e.g. the protein G-conjugated magnetic beads or the anti-human IgM antibody conjugated magnetic beads) are mixed for 10 minutes, and then the magnetic beads are washed twice using a magnetic cylinder to remove non-specific binding molecules. Next, add the CHIK VLP-Lucia and mix for 10 minutes, and then the magnetic beads are washed twice using a magnetic cylinder to remove non-specific binding molecules. The washed magnetic beads are then placed into the tube with substrate for Lucia, and the light signals can be directly measured by the smartphone.

Compared to the method that mixing the sample, the magnetic beads and the CHIK VLP-Lucia simultaneously (as shown in FIG. 7 and FIG. 15), the improved method shown in FIG. 16 is to mix the sample and the magnetic beads first, and then add the CHIK VLP-Lucia for a second mix. Although the mixing time is increased by 10 minutes, it can increase the specific bindings between antigens and antibodies and eliminate non-specific bindings, thereby strengthening the light signal from the reaction of the luciferase and the substrate. This allows the light signal to be detected directly using the smartphone without the need for other special analytical equipment, and the test results can be obtained within 25 minutes, making it possible to achieve rapid on-site testing for POCT.

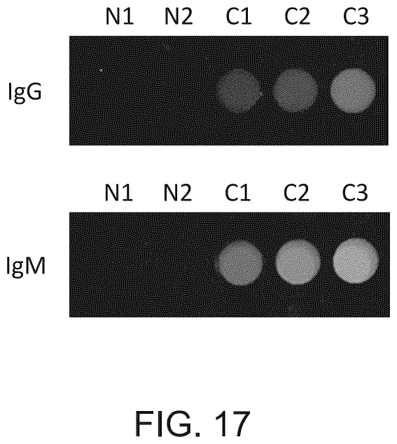

The present disclosure further verified the effectiveness of this improved method for smartphone detection. One serum sample each from low, medium, and high antibody titer groups among CHIKV patients were selected as positive controls (C1, C2 and C3) and two normal sera were randomly selected as negative controls (N1 and N2). Simulated whole blood samples were prepared by mixing 10 μl of the whole blood with 5 μl of the serum samples from CHIKV patients or normal individuals for the CHIK VLP-Lucia LIPS assay. FIG. 17 shows the smartphone detection performance of the CHIK VLP-Lucia LIPS assay. As shown in FIG. 17, the smartphones can effectively detect the light signal from the LIPS assay, and the measured light intensity is proportional to the antibody concentration. Therefore, the smartphone can be used as a quantitative real-time detection tool for the CHIK VLP-Lucia LIPS assay.

Except preparing the CHIK VLP-Lucia for the LIPS assay to detect CHIKV antibodies, the present disclosure has also successfully prepared other virus-like particles for different mosquito-borne viruses which simultaneously express Lucia as well. Common mosquito-borne viruses include alphaviruses and flaviviruses. Further, the VLP-Lucia preparation of the present disclosure can also be applied to alphaviruses and flaviviruses that are not mosquito-borne viruses. Alphaviruses include but are not limited to Chikungunya virus, Venezuelan equine encephalitis virus (VEEV), Eastern equine encephalitis virus (EEEV), Western equine encephalitis virus (WEEV), Mayaro virus (MAYV), Ross River virus (RRV), Barmah forest virus (BFV), O'nyong-nyong virus (ONNV), and Sindbis virus (SINV). Flaviviruses include but are not limited to Dengue virus (DENV, which has four serotypes: DENV1, DENV2, DENV3 and DENV4), Japanese encephalitis virus (JEV), Zika virus (ZIKV), West Nile virus (WNV), Yellow fever virus (YFV), and Hepatitis C virus.

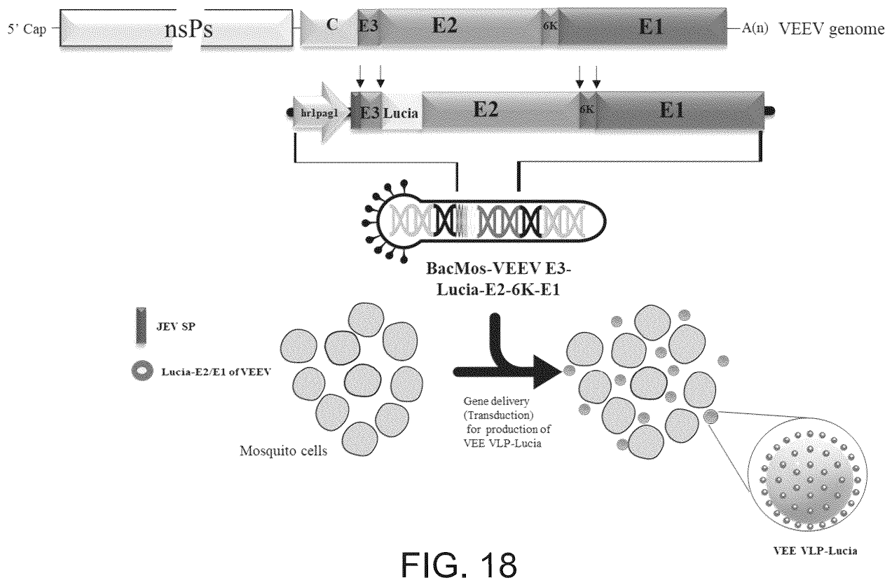

FIG. 18 shows the preparation method of the VEE VLP-Lucia. The genome structure of the VEEV is similar to that of the CHIKV. The genetic recombination design is also to insert the Lucia gene between the E2 gene and the E3 gene of the VEEV (i.e. in-frame insertion at junction of E3-E2), delete the C gene and add a mosquito promoter hr1pag1 and a signal peptide JEV SP to form a gene cassette of hr1pag1-JEV SP-VEEV E3-Lucia-E2-6K-E1. The gene cassette was recombined with the baculovirus gene, and then the recombinant baculovirus (represented by BacMos-VEEV E3-Lucia-E2-6K-E1) was transduced into mosquito cells for gene delivery, thereby inducing the expression of the VEE VLP-Lucia. By antibody staining, western blot, luciferase activity analysis and TEM analysis, it was confirmed that the VEE VLP-Lucia, which displays the fusion protein of the VEEV structural proteins and the luciferase protein, was successfully expressed, assembled and secreted through the BacMos system, and can be used for LIPS assay to detect VEEV antibodies.

In an embodiment, the recombinant sequence of JEV SP-VEEV E3-Lucia-E2-6K-E1 can be created by first synthesizing the individual sequence segments, and then combining the segments to form the complete recombinant sequence of JEV SP-VEEV E3-Lucia-E2-6K-E1 (SEQ ID NO. 5). In the recombinant sequence, the base positions 15 to 3632 are encoding sequence, which encodes 1206 amino acids (SEQ ID NO. 6). In addition, during the protein expression processing, the E3 protein and the 6K protein were cleaved, so the secreted product VEE VLP-Lucia included the Lucia-VEEV E2 protein (SEQ ID NO. 7) and the VEEV E1 protein (SEQ ID NO. 8).

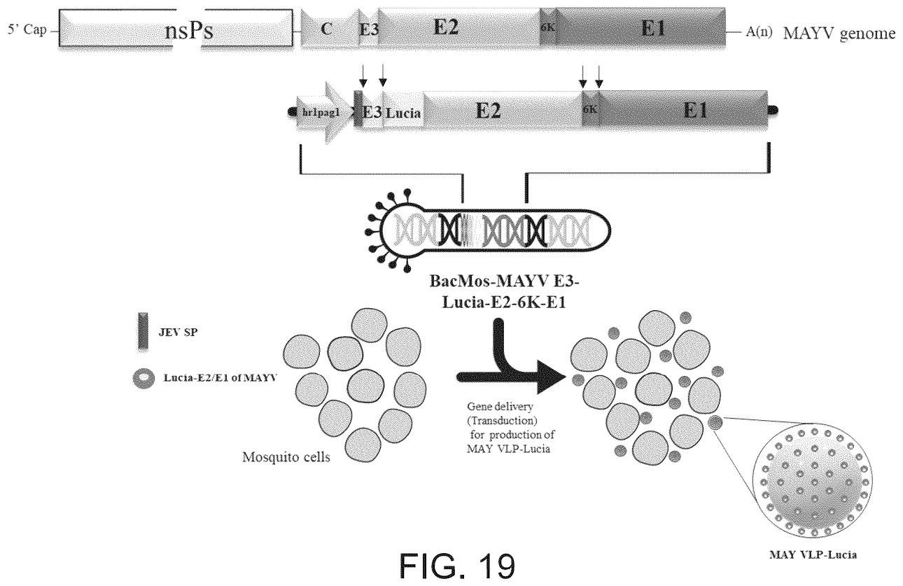

FIG. 19 shows the preparation method of the MAY VLP-Lucia. The genome structure of the MAYV is similar to that of the CHIKV. The genetic recombination design is also to insert the Lucia gene between the E2 gene and the E3 gene of the MAYV (i.e. in-frame insertion at junction of E3-E2), delete the C gene and add a mosquito promoter hr1pag1 and a signal peptide JEV SP to form a gene cassette of hr1pag1-JEV SP-MAYV E3-Lucia-E2-6K-E1. The gene cassette was recombined with the baculovirus gene, and then the recombinant baculovirus (represented by BacMos-MAYV E3-Lucia-E2-6K-E1) was transduced into mosquito cells for gene delivery, thereby inducing the expression of the MAY VLP-Lucia. By antibody staining, western blot, luciferase activity analysis and TEM analysis, it was confirmed that the MAY VLP-Lucia, which displays the fusion protein of the MAYV structural proteins and the luciferase protein, was successfully expressed, assembled and secreted through the BacMos system, and can be used for LIPS assay to detect MAYV antibodies.

In an embodiment, the recombinant sequence of JEV SP-MAYV E3-Lucia-E2-6K-E1 can be created by first synthesizing the individual sequence segments, and then combining the segments to form the complete recombinant sequence of JEV SP-MAYV E3-Lucia-E2-6K-E1 (SEQ ID NO. 9). In the recombinant sequence, the base positions 15 to 3635 are encoding sequence, which encodes 1207 amino acids (SEQ ID NO. 10). In addition, during the protein expression processing, the E3 protein and the 6K protein were cleaved, so the secreted product MAY VLP-Lucia included the Lucia-MAYV E2 protein (SEQ ID NO. 11) and the MAYV E1 protein (SEQ ID NO. 12).

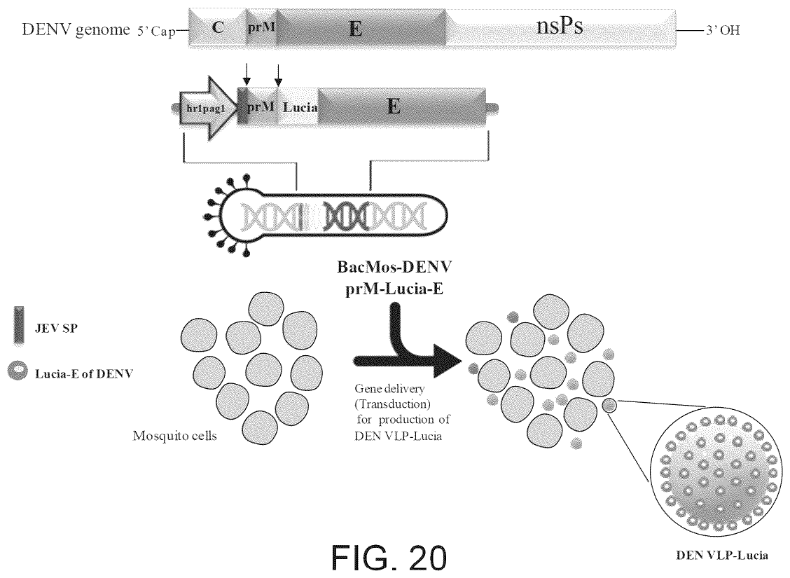

FIG. 20 shows the preparation method of the DEN VLP-Lucia. The DENV genome includes nonstructural protein gene (nsPs) and structural protein gene. The structural proteins include prM precursor membrane protein, E envelope protein, and C capsid protein. The genetic recombination design is to insert the Lucia gene between the prM gene and the E gene of the DENV (i.e. in-frame insertion at junction of prM-E), delete the C gene and add a mosquito promoter hr1pag1 and a signal peptide JEV SP to form a gene cassette of hr1pag1-JEV SP-DENV prM-Lucia-E. The gene cassette was recombined with the baculovirus gene, and then the recombinant baculovirus (represented by BacMos-DENV prM-Lucia-E) was transduced into mosquito cells for gene delivery, thereby inducing the expression of the DEN VLP-Lucia. By antibody staining, western blot, luciferase activity analysis and TEM analysis, it was confirmed that the DEN VLP-Lucia, which displays the fusion protein of the DENV structural proteins and the luciferase protein, was successfully expressed, assembled and secreted through the BacMos system, and can be used for LIPS assay to detect DENV antibodies.

In an embodiment, the recombinant sequence of JEV SP-DENV prM-Lucia-E can be created by first synthesizing the individual sequence segments, and then combining the segments to form the complete recombinant sequence of JEV SP-DENV prM-Lucia-E (SEQ ID NO. 13). In the recombinant sequence, the base positions 35 to 2677 are encoding sequence, which encodes 804 amino acids (SEQ ID NO. 14). In addition, during the protein expression processing, the prM protein was cleaved, so the secreted product DEN VLP-Lucia included the Lucia-DENV E protein (SEQ ID NO. 15).

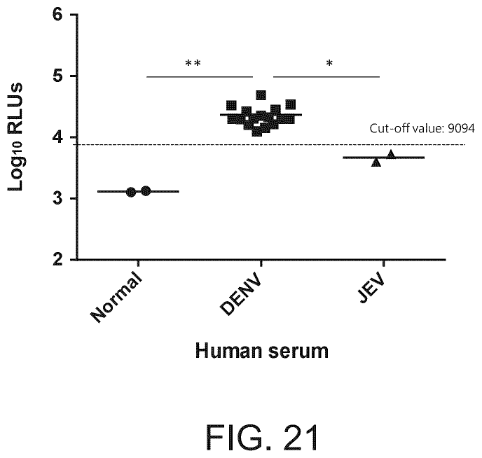

Multiple clinical samples were further used to analyze the detection efficacy of the DEN VLP-Lucia LIPS assay. 20 laboratory-confirmed serum samples were tested. These samples included 16 from DENV-infected individuals, 2 from JEV-infected individuals, and 2 from normal individuals. FIG. 21 shows the LIPS assay results to the 20 samples by the DEN VLP-Lucia provided in the present disclosure. According to the test results, the DEN VLP-Lucia LIPS assay effectively detected 16 DENV-positive samples with a detection rate of 100%.

From the above, the present disclosure provides the novel nanobiosensor including the virus-like particle and the luciferase. The virus-like particle is assembled by the structural protein(s) of the virus. The luciferase forms a fusion protein with the structural protein of the virus and is displayed on the surface of the virus-like particle. The virus-like particle can bind to specific antibodies against the virus, and a rapid antibody detection of the virus can be achieved by immunoassay through the luciferase. This nanobiosensor of VLP-Lucia is a luminescent nanoparticle and can be combined with LIPS assay for detecting virus antibodies, and thus is an effective diagnostic tool.

The present disclosure also provides a preparation method of the nanobiosensor. The method is characterized by inserting the luciferase gene into the structural protein gene of the virus, and using the BacMos system to express, assemble and secrete the virus-like particle. Particularly, the luciferase protein and the structural protein of the virus form the fusion protein, which is displayed on the surface of the virus-like particle.

On the other hand, the present disclosure further provides a detection method of virus antibodies. First, the aforesaid nanobiosensor and magnetic beads for capturing human IgG or IgM are provided. The nanobiosensor, the magnetic beads and the sample are mixed, so that the magnetic beads captures the immune complex formed by the virus antibody and the nanobiosensor. After the magnetic beads are washed twice using a magnetic cylinder, the substrate of the luciferase is added, and then the light signal is further detected to diagnose the presence of virus antibodies in the sample. Preferably, the sample includes but not limited to serum, blood, saliva, and monoclonal antibody.

Furthermore, in addition to using the structural protein(s) of the virus as antigens to detect the virus antibodies, since the VLP-Lucia prepared in the present disclosure has a stable structure, it may be further applied to detect other antibodies by adding other antigens thereon. That is, during the genetic recombination design, the other antigen gene is also inserted as inserting the Lucia gene, so that the other antigen forms a fusion protein with the structural protein and displays on the surface of the VLP. For example, during the genetic recombination design of CHIK VLP-Lucia, a DENV antigen gene is also inserted into the structural protein gene of the CHIKV as inserting the Lucia gene, so that the Lucia, the structural protein of the CHIKV and the antigen protein of the DENV are co-displayed on the surface of the VLP. Accordingly, the antigen protein of the DENV can be used to bind with specific antibodies against the DENV in the sample for DENV detection. Therefore, in the design of the present disclosure, the VLP-Lucia can be further applied to immunoassays of other additional antigens.

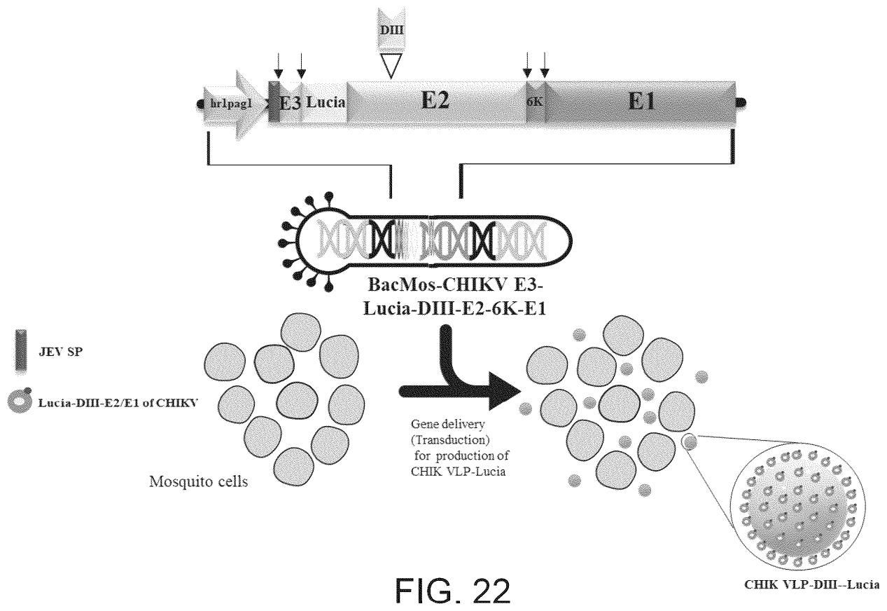

In an embodiment, since the domain III (DIII) of the DENV envelop protein is the site where the virus binds to the cell receptor, the DENV DIII protein is selected as the antigen protein and co-displayed with the Lucia on the surface of the VLP. FIG. 22 shows the preparation method of the CHIK VLP-DIII-Lucia. Compared to CHIK VLP-Lucia, the genetic recombination design of the CHIK VLP-DIII-Lucia additionally inserts the DIII gene into the CHIKV E2 gene, and expresses the CHIK VLP-DIII-Lucia having the fusion protein of E1/E2, Lucia and DIII displayed on the surface thereof.

In an embodiment, the recombinant sequence of JEV SP-CHIKV E3-Lucia-E2-DIII-6K-E1 can be created by first synthesizing the individual sequence segments, and then combining the segments to form the complete recombinant sequence of JEV SP-CHIKV E3-Lucia-E2-DIII-6K-E1 (SEQ ID NO. 16). In the recombinant sequence, the base positions 29 to 4012 are encoding sequence, which encodes 1328 amino acids (SEQ ID NO. 17). In addition, during the protein expression processing, the E3 protein and the 6K protein were cleaved, so the secreted product CHIK VLP-DIII-Lucia included the Lucia-CHIKV E2-DIII protein (SEQ ID NO. 18) and the CHIKV E1 protein (SEQ ID NO. 19).

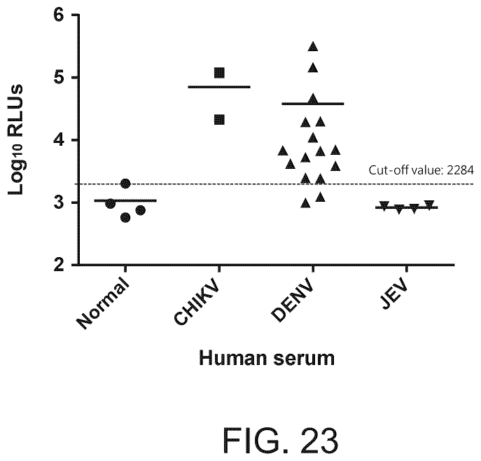

Multiple clinical samples were further used to analyze the detection efficacy of the CHIK VLP-DIII-Lucia LIPS assay. 26 laboratory-confirmed serum samples were tested. These samples included 2 from CHIKV-infected individuals, 16 from DENV-infected individuals, 4 from JEV-infected individuals, and 4 from normal individuals. FIG. 23 shows the LIPS assay results to the 26 samples by the CHIK VLP-DIII-Lucia provided in the present disclosure. According to the test results, the CHIK VLP-DIII-Lucia LIPS assay effectively detected 2 CHIKV-positive samples, and detected 14 DENV-positive samples with a detection rate of 87%. Further, no JEV-positive samples were detected, which implies the CHIK VLP-DIII-Lucia LIPS assay can avoid cross-interference from non-DENV infections such as JEV.

In conclusion, the present disclosure has developed the novel nanobiosensor of VLP-Lucia. The luciferase protein and the structural protein of the virus form the fusion protein, which is displayed on the surface of the virus-like particle. This nanobiosensor is an enzyme-functionalized virus-like particle having biocompatibility and enzymatic properties, and can bind with the specific antibodies against the virus. By using the luciferase immunoprecipitation system for immunoassay, the nanobiosensor exhibits excellent antibody biosensing capabilities to rapidly and accurately distinguish between positive and negative samples within 15 minutes without prior sample pretreatment. This highly sensitive and rapid luminometric biosensing platform has the potential for directly measuring virus antibodies in whole blood samples and saliva samples in POCT settings. Therefore, the present disclosure provides a simple, accurate, and rapid high-throughput antibody diagnostic tool platform, which holds great promise as a highly specific serological assay for global vaccination program testing, epidemiological studies, and immune responses and immunity duration monitoring. Significantly, this study not only introduces the VLP-Lucia as a promising luminescent nanoprobe for antibody biosensing but also opens a new avenue for simple, sensitive, and robust antibody detection in disease diagnosis and clinical applications.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A nanobiosensor, comprising:
a virus-like particle assembled by a structural protein of a virus; and
a luciferase forming a fusion protein with the structural protein of the virus and displayed on the surface of the virus-like particle,
wherein the virus-like particle is capable of capturing specific antibodies against the virus, and the luciferase is analyzed through a luciferase immunoprecipitation system, which enables a rapid detection of the specific antibodies against the virus.

2. The nanobiosensor according to claim 1, wherein the virus-like particle does not contain a capsid protein of the virus.

3. The nanobiosensor according to claim 1, wherein the virus is a mosquito-borne virus.

4. The nanobiosensor according to claim 1, wherein the virus is an alphavirus or a flavivirus.

5. The nanobiosensor according to claim 4, wherein the alphavirus comprises Chikungunya virus, Venezuelan equine encephalitis virus, Eastern equine encephalitis virus, Western equine encephalitis virus, Mayaro virus, Ross River virus, Barmah forest virus, O'nyong-nyong virus, and Sindbis virus.

6. The nanobiosensor according to claim 4, wherein the flavivirus comprises Dengue virus, Japanese encephalitis virus, Zika virus, West Nile virus, Yellow fever virus, and Hepatitis C virus.

7. The nanobiosensor according to claim 1, wherein the luciferase is Lucia luciferase.

8. The nanobiosensor according to claim 1, wherein the nanobiosensor comprises proteins of SEQ ID NO. 3 and SEQ ID NO. 4.

9. The nanobiosensor according to claim 1, wherein the nanobiosensor comprises proteins of SEQ ID NO. 7 and SEQ ID NO. 8.

10. The nanobiosensor according to claim 1, wherein the nanobiosensor comprises proteins of SEQ ID NO. 11 and SEQ ID NO. 12.

11. The nanobiosensor according to claim 1, wherein the nanobiosensor comprises a protein of SEQ ID NO. 15.

12. The nanobiosensor according to claim 1, wherein the nanobiosensor comprises proteins of SEQ ID NO. 18 and SEQ ID NO. 19.

13. The nanobiosensor according to claim 1, wherein the nanobiosensor further comprises an additional antigen, which forms a fusion protein with the luciferase and is displayed on the surface of the virus-like particle.

14. A preparation method of a nanobiosensor, characterized by inserting a luciferase gene into a structural protein gene of a virus, and using a baculovirus/mosquito system to express, assemble and secrete a virus-like particle, wherein an expressed luciferase protein and an expressed structural protein of the virus form a fusion protein displayed on a surface of the virus-like particle.

15. A detection method of virus antibodies, comprising steps of:
(a) providing a nanobiosensor and magnetic beads for capturing human IgG or IgM, wherein the nanobiosensor comprises a virus-like particle assembled by a structural protein of a virus and a luciferase forming a fusion protein with the structural protein of the virus and displayed on the surface of the virus-like particle;
(b) mixing the nanobiosensor, the magnetic beads and a sample;
(c) washing the magnetic beads to remove non-specific bindings; and
(d) adding a substrate of the luciferase and detecting a light signal.

16. The detection method of the virus antibodies according to claim 15, wherein the magnetic beads are protein G-conjugated magnetic beads or anti-human IgM antibody conjugated magnetic beads.

17. The detection method of the virus antibodies according to claim 15, wherein the steps (b) and (c) further comprise steps of:
(b1) mixing the magnetic beads and the sample;
(c1) washing the magnetic beads to remove non-specific bindings;
(b2) adding the nanobiosensor for further mixing; and
(c2) washing the magnetic beads to remove non-specific bindings.

18. The detection method of the virus antibodies according to claim 15, wherein in the step (d), the light signal is detected by a smartphone or a portable luminometer.

19. The detection method of the virus antibodies according to claim 15, wherein in the step (b), the sample comprises serum, blood, saliva, and monoclonal antibody.


SEQUENCE LISTING (19 sequences)

SEQ ID NO: 1 - DNA, 3714 bp, synthetic construct
  source 1..3714: mol_type = other DNA
gagtcgtgttcataccgcatatgccaccatgggtggcaacgagggttccatcatgtggctggcttccctggctgtggtcatcgcttgcgccggtgctatgagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcacccgaaagccaaccgaaatcaacgaggacctgaacatcgctgccgtggcttccaacttcgccaccactgacctggaaaccgacctgttcactaactgggagaccatgaacgtcatcagcaccgacactgaacaggtgaacaccgacgctgaccgtggaaagctgcctggcaagaagctgcccccagacgtcctgagagagctggaagctaacgctaggagagctggatgcactaggggatgcctgatctgcctgtcccacatcaagtgcacccctaagatgaagaagttcatccccggaaggtgccacacttacgagggtgaaaaggagagcgctcagggcggaatcggcgaagccatcgtggacatccctgagatccccggattcaaggacaaggagcccctggaccagttcatcgctcaggtcgacctgtgcgccgactgcaccactggttgcctgaagggcctggctaacgtgcagtgctctgacctgctgaagaagtggctgccacagcgctgcaccactttcgcttcaaagatccagggtcgtgtggacaagatcaagggactggctggagacaggggcggtggctcagactctagaaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcactaacttgacaattaagtatgaagggcggccgcagtagatgccgaccgaacaagagctga

SEQ ID NO: 2 - protein, 1209 aa, synthetic construct
MGGNEGSIMWLASLAVVIACAGAMSLAIPVMCLLANTTFPCSQPPCTPCCYEKEPEETLRMLEDNVMRPGYYQLLQASLTCSPHRQRRSTRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSDSRNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKAATYQEAAIYLWNEQQPLFWLQALIPLAALIVLCNCLRLLPCCCKTLAFLAVMSVGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 3 - protein, 621 aa, synthetic construct
STRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSDSRNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA

SEQ ID NO: 4 - protein, 439 aa, synthetic construct
YEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 5 - DNA, 3670 bp, synthetic construct
  source 1..3670: mol_type = other DNA
atgcccgggccaccatgggtggcaacgagggttccatcatgtggctggcttccctggctgtggtcatcgcttgcgccggtgctatgagcctggtgaccactatgtgcctgctggctaacgtcactttcccctgcgcccagcctcccatctgctacgaccgcaagccagctgagaccctggccatgctgtccgtgaacgtcgacaacccaggatacgacgagctgctggaagctgccgtcaagtgccctggtaggaagcgccgtagcactcgaaagccaaccgaaatcaacgaggacctgaacatcgctgccgtggcttccaacttcgccaccactgacctggaaaccgacctgttcactaactgggagaccatgaacgtcatcagcaccgacactgaacaggtgaacaccgacgctgaccgtggaaagctgcctggcaagaagctgcccccagacgtcctgagagagctggaagctaacgctaggagagctggatgcactaggggatgcctgatctgcctgtcccacatcaagtgcacccctaagatgaagaagttcatccccggaaggtgccacacttacgagggtgaaaaggagagcgctcagggcggaatcggcgaagccatcgtggacatccctgagatccccggattcaaggacaaggagcccctggaccagttcatcgctcaggtcgacctgtgcgccgactgcaccactggttgcctgaagggcctggctaacgtgcagtgctctgacctgctgaagaagtggctgccacagcgctgcaccactttcgcttcaaagatccagggtcgtgtggacaagatcaagggactggctggagacaggggcggtggctcaggagggtcgagcactgaggaactgttcaacgagtacaagctgaccagaccctacatggctcgctgcatccgttgcgccgtgggttcttgccactcaccaatcgctatcgaagccgtgaagtccgacggacacgacggttacgtccgtctgcagacctccagccagtacggactggactcttcaggtaacctgaagggcaggactatgagatacgacatgcacggtaccatcaaggagatccctctgcaccaggtgtccctgtacacttctaggccatgccacatcgtggacggacacggttacttcctgctggctaggtgcccagctggtgactctatcaccatggaattcaagaaggactcagtccgccacagctgctctgtgccttacgaggtcaagttcaaccccgtgggtcgtgaactgtacacccacccacctgagcacggcgtggaacaggcttgccaggtctacgctcacgacgcccagaaccgcggtgcttacgtcgagatgcacctgcctggatctgaagtggactccagcctggtctcactgtccggatcttcagtgaccgtcactcccccagacggtacttcagctctggtggagtgcgaatgcggtggcaccaagatctccgagactatcaacaagaccaagcagttcagccagtgcactaagaaggaacagtgccgcgcttaccgtctgcagaacgacaagtgggtgtacaactctgacaagctgccaaaggctgctggtgctaccctgaagggaaagctgcacgtgccattcctgctggctgacggcaagtgcactgtccctctggcccccgagccaatgatcaccttcggattcaggagcgtgtctctgaagctgcacccaaagaaccctacttacctgatcaccagacagctggctgacgaaccccactacactcacgagctgatctccgaaccagccgtgaggaacttcaccgtcactgagaagggttgggaattcgtctggggcaaccaccctcccaagagattctgggctcaggaaactgctcctggaaacccacacggtctgcctcacgaagtgatcactcactactaccaccgttaccccatgtccaccatcctgggtctgagcatctgcgctgccatcgctactgtgtctgtcgctgcctcaacctggctgttctgcaggtccagagtcgcttgcctgactccatacaggctgacccctaacgccagaatccccttctgcctggctgtgctgtgctgcgccaggactgctagagccgagaccacttgggaaagcctggaccacctgtggaacaacaaccagcagatgttctggatccagctgctgatccctctggctgccctgatcgtggtcactaggctgctgaggtgcgtgtgctgcgtggtccccttcctggtcatggctggtgctgctggtgctggagcttacgagcacgctaccactatgccctcccaggccggaatcagctacaacactatcgtgaacagggctggttacgcccctctgcccatctccatcaccccaactaagatcaagctgatccctaccgtgaacctggaatacgtcacttgtcactacaagaccggcatggactccccagctatcaagtgctgcggaagccaggagtgcacccctacttacagacccgacgaacagtgcaaggtgttcaccggcgtctaccctttcatgtggggaggtgcctactgcttctgcgacaccgagaacactcaggtgtctaaggcttacgtcatgaagtcagacgactgcctggccgaccacgctgaagcctacaaggctcacactgcctccgtgcaggctttcctgaacatcaccgtgggcgagcacagcatcgtcaccactgtgtacgtcaacggagaaactcccgtgaacttcaacggagtcaagatcaccgctggtcccctgtctaccgcctggactccattcgacaggaagatcgtccagtacgctggagagatctacaactacgacttcccagaatacggtgctggtcaacctggagctttcggagacatccagtcacgcactgtgtccagctctgacctgtacgctaacaccaacctggtgctgcagcgtccaaaggctggtgccatccacgtcccatacacccaggccccttctggcttcgagcagtggaagaaggacaaggctccatcactgaagttcactgcccctttcggttgcgagatctacaccaaccctatccgcgctgaaaactgcgctgtgggatccatcccactggctttcgacatcccagacgccctgttcactcgtgtgtctgagacccccactctgtcagctgccgagtgcaccctgaacgaatgcgtgtactcatccgacttcggcggaatcgctactgtcaagtactcagcctccaagagcggaaagtgcgctgtgcacgtcccttctggaactgctactctgaaggaggctgctgtggagctgaccgaacagggctccgctactatccacttcagcaccgccaacatccaccccgaattccgcctgcagatctgcacttcttacgtgacctgcaagggagactgccacccacctaaggaccacatcgtgacccacccacagtaccacgctcagaccttcactgctgccgtgtcaaagaccgcctggacttggctgacctctctgctgggtggctcagctgtgatcatcatcatcggcctggtcctggctactatcgtggccatgtacgtcctgaccaaccagaagcacaactgagcggccgcagtagatgccgaccgaacaagagctga

SEQ ID NO: 6 - protein, 1206 aa, synthetic construct
MGGNEGSIMWLASLAVVIACAGAMSLVTTMCLLANVTFPCAQPPICYDRKPAETLAMLSVNVDNPGYDELLEAAVKCPGRKRRSTRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSGGSSTEELFNEYKLTRPYMARCIRCAVGSCHSPIAIEAVKSDGHDGYVRLQTSSQYGLDSSGNLKGRTMRYDMHGTIKEIPLHQVSLYTSRPCHIVDGHGYFLLARCPAGDSITMEFKKDSVRHSCSVPYEVKFNPVGRELYTHPPEHGVEQACQVYAHDAQNRGAYVEMHLPGSEVDSSLVSLSGSSVTVTPPDGTSALVECECGGTKISETINKTKQFSQCTKKEQCRAYRLQNDKWVYNSDKLPKAAGATLKGKLHVPFLLADGKCTVPLAPEPMITFGFRSVSLKLHPKNPTYLITRQLADEPHYTHELISEPAVRNFTVTEKGWEFVWGNHPPKRFWAQETAPGNPHGLPHEVITHYYHRYPMSTILGLSICAAIATVSVAASTWLFCRSRVACLTPYRLTPNARIPFCLAVLCCARTARAETTWESLDHLWNNNQQMFWIQLLIPLAALIVVTRLLRCVCCVVPFLVMAGAAGAGAYEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKITAGPLSTAWTPFDRKIVQYAGEIYNYDFPEYGAGQPGAFGDIQSRTVSSSDLYANTNLVLQRPKAGAIHVPYTQAPSGFEQWKKDKAPSLKFTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 7 - protein, 625 aa, synthetic construct
STRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSGGSSTEELFNEYKLTRPYMARCIRCAVGSCHSPIAIEAVKSDGHDGYVRLQTSSQYGLDSSGNLKGRTMRYDMHGTIKEIPLHQVSLYTSRPCHIVDGHGYFLLARCPAGDSITMEFKKDSVRHSCSVPYEVKFNPVGRELYTHPPEHGVEQACQVYAHDAQNRGAYVEMHLPGSEVDSSLVSLSGSSVTVTPPDGTSALVECECGGTKISETINKTKQFSQCTKKEQCRAYRLQNDKWVYNSDKLPKAAGATLKGKLHVPFLLADGKCTVPLAPEPMITFGFRSVSLKLHPKNPTYLITRQLADEPHYTHELISEPAVRNFTVTEKGWEFVWGNHPPKRFWAQETAPGNPHGLPHEVITHYYHRYPMSTILGLSICAAIATVSVAASTWLFCRSRVACLTPYRLTPNARIPFCLAVLCCARTARA

SEQ ID NO: 8 - protein, 442 aa, synthetic construct
YEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKITAGPLSTAWTPFDRKIVQYAGEIYNYDFPEYGAGQPGAFGDIQSRTVSSSDLYANTNLVLQRPKAGAIHVPYTQAPSGFEQWKKDKAPSLKFTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 9 - DNA, 3673 bp, synthetic construct
  source 1..3673: mol_type = other DNA
atgcccgggccaccatgggtggcaacgagggtagcatcatgtggctggcctctctggctgtggtcatcgcttgcgccggtgctatggccgcttctaccgtgactgctatgtgcctgctgactaacatctcattcccctgcttccagccatcctgcgccccttgctgctacgagaagggtcccgaaccaaccctgcgtatgctggaggaaaacgtcaactccgagggctactacgacctgctgcacgccgctgtgtactgccgcaactcttcaaggagcaagagatctactcgcaagccaaccgaaatcaacgaggacctgaacatcgctgccgtggcttccaacttcgccaccactgacctggaaaccgacctgttcactaactgggagaccatgaacgtcatcagcaccgacactgaacaggtgaacaccgacgctgaccgtggaaagctgcctggcaagaagctgcccccagacgtcctgagagagctggaagctaacgctaggagagctggatgcactaggggatgcctgatctgcctgtcccacatcaagtgcacccctaagatgaagaagttcatccccggaaggtgccacacttacgagggtgaaaaggagagcgctcagggcggaatcggcgaagccatcgtggacatccctgagatccccggattcaaggacaaggagcccctggaccagttcatcgctcaggtcgacctgtgcgccgactgcaccactggttgcctgaagggcctggctaacgtgcagtgctctgacctgctgaagaagtggctgccacagcgctgcaccactttcgcttcaaagatccagggtcgtgtggacaagatcaagggactggctggagacaggggcggtggctcatctactgccaaccacttcaacgcttacaagctgacccgtccatacgtggcctactgcgctgactgcggtatgggccactcatgccactcccctgctatgatcgaaaacatccaggctgacgccaccgacggaactctgaagatccagttcgcctcacagatcggtctgaccaagactgacacccacgaccacactaagatccgttacgctgagggacacgacatcgctgaagctgctagatccaccctgaaggtccactccagctctgagtgcactgtgaccggtactatgggccacttcatcctggccaagtgcccacctggagaggctatcagcgtgtctttcgtcgacagcaagaacgaacaccgcacttgccgtatcgctttccaccacgagcagaggctgatcggaagggaacgtttcaccgtgcgtcctcaccacggaatcgagctgccctgcaccacttaccagctgaccactgccgaaacttctgaggaaatcgacatgcacatgcccccagacatcccagaccgcaccatcctgagccagcagtctggcaacgtgaagatcactgtcaacggaaggaccgtgagatactcatgctcctgcggatcaaagccttccggtaccactaccactgacaagactatcaacagctgcaccgtcgacaagtgccaggcttacgtgaccagccacactaagtggcagttcaactctcctttcgtccccagggccgagcaggctgaaagaaagggaaaggtgcacatccctttccccctgatcaacaccacttgcagggtgcctctggctcctgaggctctcgtgaggtctggaaagcgtgaagctaccctgtccctgcaccccatccacccaactctgctgtcttaccgtactctgggtgctgagcctgtgttcgacgaacagtggatcaccgctcagactgaggtcaccatcccagtgcctgtcgagggcgtggaataccagtggggaaaccacaagccacagcgtctgtggagccagctgaccactgaaggaaaggctcacggctggccccacgagatcatcgaatactactacggcctgcacccaaccatcactatcgtggtcgtgatcgctgtctctgtcgtggtcctgctgtcactggccgcttccgtgtacatgtgcgtggtcgcccgcaacaagtgcctgactccctacgccctgaccccaggtgctgtggtccctgtgaccatcggcgtgctctgctgcgctcctaaggctcacgctgcttcattcgccgagggcatggcttacctgtgggacaacaaccagtccatgttctggatggaactgaccggacccctggccctgctgatcctgaccacttgctgcgctagaagcctgttctcttgctgcaagggaagcttcctggtcgctatgtcaatcggttccgctgtggcctctgcttacgagcacaccgccatcatccctaaccaggtcggtttcccctacaaggcccacgtggctagggaaggctactcacccctgaccctgcagatgcaggtggtcgagacttccctggaaccaaccctgaacctggagtacatcacttgcgactacaagaccaaggtcccctccccatacgtgaagtgctgcggtactgccgagtgccgtacccaggacaagcctgaatacaagtgcgctgtgttcactggcgtctaccccttcatgtggggcggagcctactgcttctgcgacagcgaaaacacccagatgtctgaggcctacgtcgaaagggctgacgtgtgcaagcacgaccacgccgctgcctacagggctcacactgcttcactgagagctaagatcaaagtgacttacggaaccgtgaaccagaccgtcgaggcctacgtgaacggtgaccacgccgtcactatcgctggtaccaagttcatcttcggcccagtgtccactgcttggacccctttcgacaccaagatcgtggtctacaagggtgaagtgtacaaccaggacttccctccctacggagctggtcagcccggaagattcggtgacatccagtcaaggactctggactccagagacctgtacgctaacactggactgaagctggctaggccagctgctggaaacatccacgtcccctacacccagactccaagcggcttcaagacttggcagaaggacagggactctccactgaacgccaaggctcctttcggatgcatcatccagaccaacccagtgagagccatgaactgcgctgtcggtaacatccctgtgtcaatggacatcgccgactccgctttcactcgtctgaccgacgctcctgtcatcagcgagctgacctgcactgtgtctacctgcactcactcatccgacttcggtggcatcgccgtcctgagctacaaggtggagaagtctggccgctgcgacatccacagccactctaacgtggctgtcctgcaggaagtctcaatcgagactgaaggacgttccgtgatccacttctctactgcttctgctgctccaagcttcgtggtctctgtctgcagctctagggccacctgcactgctaagtgcgagccacctaaggaccacgtggtcacttaccctgctaaccacaacggtgtgaccctgcccgacctgtcatccactgccatgacctgggctcaacacctcgccggaggtgtcggactgctgatcgccctggctgtgctgtccctggctatcgtgacttgcgtcaccctgaggagataagcggccgcagtagatgccgaccgaacaagagctga

SEQ ID NO: 10 - protein, 1207 aa, synthetic construct
MGGNEGSIMWLASLAVVIACAGAMAASTVTAMCLLTNISFPCFQPSCAPCCYEKGPEPTLRMLEENVNSEGYYDLLHAAVYCRNSSRSKRSTRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSSTANHFNAYKLTRPYVAYCADCGMGHSCHSPAMIENIQADATDGTLKIQFASQIGLTKTDTHDHTKIRYAEGHDIAEAARSTLKVHSSSECTVTGTMGHFILAKCPPGEAISVSFVDSKNEHRTCRIAFHHEQRLIGRERFTVRPHHGIELPCTTYQLTTAETSEEIDMHMPPDIPDRTILSQQSGNVKITVNGRTVRYSCSCGSKPSGTTTTDKTINSCTVDKCQAYVTSHTKWQFNSPFVPRAEQAERKGKVHIPFPLINTTCRVPLAPEALVRSGKREATLSLHPIHPTLLSYRTLGAEPVFDEQWITAQTEVTIPVPVEGVEYQWGNHKPQRLWSQLTTEGKAHGWPHEIIEYYYGLHPTITIVVVIAVSVVVLLSLAASVYMCVVARNKCLTPYALTPGAVVPVTIGVLCCAPKAHAASFAEGMAYLWDNNQSMFWMELTGPLALLILTTCCARSLFSCCKGSFLVAMSIGSAVASAYEHTAIIPNQVGFPYKAHVAREGYSPLTLQMQVVETSLEPTLNLEYITCDYKTKVPSPYVKCCGTAECRTQDKPEYKCAVFTGVYPFMWGGAYCFCDSENTQMSEAYVERADVCKHDHAAAYRAHTASLRAKIKVTYGTVNQTVEAYVNGDHAVTIAGTKFIFGPVSTAWTPFDTKIVVYKGEVYNQDFPPYGAGQPGRFGDIQSRTLDSRDLYANTGLKLARPAAGNIHVPYTQTPSGFKTWQKDRDSPLNAKAPFGCIIQTNPVRAMNCAVGNIPVSMDIADSAFTRLTDAPVISELTCTVSTCTHSSDFGGIAVLSYKVEKSGRCDIHSHSNVAVLQEVSIETEGRSVIHFSTASAAPSFVVSVCSSRATCTAKCEPPKDHVVTYPANHNGVTLPDLSSTAMTWAQHLAGGVGLLIALAVLSLAIVTCVTLRR

SEQ ID NO: 11 - protein, 621 aa, synthetic construct
STRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSSTANHFNAYKLTRPYVAYCADCGMGHSCHSPAMIENIQADATDGTLKIQFASQIGLTKTDTHDHTKIRYAEGHDIAEAARSTLKVHSSSECTVTGTMGHFILAKCPPGEAISVSFVDSKNEHRTCRIAFHHEQRLIGRERFTVRPHHGIELPCTTYQLTTAETSEEIDMHMPPDIPDRTILSQQSGNVKITVNGRTVRYSCSCGSKPSGTTTTDKTINSCTVDKCQAYVTSHTKWQFNSPFVPRAEQAERKGKVHIPFPLINTTCRVPLAPEALVRSGKREATLSLHPIHPTLLSYRTLGAEPVFDEQWITAQTEVTIPVPVEGVEYQWGNHKPQRLWSQLTTEGKAHGWPHEIIEYYYGLHPTITIVVVIAVSVVVLLSLAASVYMCVVARNKCLTPYALTPGAVVPVTIGVLCCAPKAHA

SEQ ID NO: 12 - protein, 436 aa, synthetic construct
YEHTAIIPNQVGFPYKAHVAREGYSPLTLQMQVVETSLEPTLNLEYITCDYKTKVPSPYVKCCGTAECRTQDKPEYKCAVFTGVYPFMWGGAYCFCDSENTQMSEAYVERADVCKHDHAAAYRAHTASLRAKIKVTYGTVNQTVEAYVNGDHAVTIAGTKFIFGPVSTAWTPFDTKIVVYKGEVYNQDFPPYGAGQPGRFGDIQSRTLDSRDLYANTGLKLARPAAGNIHVPYTQTPSGFKTWQKDRDSPLNAKAPFGCIIQTNPVRAMNCAVGNIPVSMDIADSAFTRLTDAPVISELTCTVSTCTHSSDFGGIAVLSYKVEKSGRCDIHSHSNVAVLQEVSIETEGRSVIHFSTASAAPSFVVSVCSSRATCTAKCEPPKDHVVTYPANHNGVTLPDLSSTAMTWAQHLAGGVGLLIALAVLSLAIVTCVTLRR

SEQ ID NO: 13 - DNA, 2677 bp, synthetic construct
  source 1..2677: mol_type = other DNA
gagtcgtgttcataccgcatatgcccgggccaccatgggtggcaacgagggatccatcatgtggctggccagcctggctgtggtcatcgcttgcgccggtgctatgttccacctgaccactaggggaggtgagccccacatgatcgtctctaagcaggaacgtggaaagtctctgctgttcaagacctcagctggtgtgaacatgtgcactctgatcgccatggacctgggagagctgtgcgaagacaccatgacttacaagtgcccaaggatcactgaggctgaacctgacgacgtcgactgctggtgcaacgccaccgacacttgggtgacttacggtacttgctcccagactggtgaacacaggagggacaagaggtctgtggctctggctccacacgtgggtctgggcctggagacccgtactgaaacctggatgtccagcgagggcgcttggaagcagatccagagggtcgaaacctgggctctgagacacccaggattcactgtgatcgccctgttcctggcccacgctatcggcacttccatcacccagaagggaatcatcttcatcctgctgatgctggtgacccctagcatggctaagccaaccgaaatcaacgaggacctgaacatcgctgccgtggcttccaacttcgccaccactgacctggaaaccgacctgttcactaactgggagaccatgaacgtcatcagcaccgacactgaacaggtgaacaccgacgctgaccgtggaaagctgcctggcaagaagctgcccccagacgtcctgagagagctggaagctaacgctaggagagctggatgcactaggggatgcctgatctgcctgtcccacatcaagtgcacccctaagatgaagaagttcatccccggaaggtgccacacttacgagggtgaaaaggagagcgctcagggcggaatcggcgaagccatcgtggacatccctgagatccccggattcaaggacaaggagcccctggaccagttcatcgctcaggtcgacctgtgcgccgactgcaccactggttgcctgaagggcctggctaacgtgcagtgctctgacctgctgaagaagtggctgccacagcgctgcaccactttcgcttcaaagatccagggtcgtgtggacaagatcaagggactggctggagacaggggcggtggctcaatgcgctgcgtcggaatcggttctcgtgacttcgtggagggactgtcaggtgctacttgggtcgacgtggtcctggaacacggctcctgcgtgaccactatggccaaggacaagcctaccctggacatcgagctgctgaagactgaagtcaccaaccccgctatcctgaggaagctgtgcatcgaggccaagatctccaacaccactaccgacagcagatgccctacccagggtgaagctactctggtggaggaacaggacgccaacttcgtctgcaggagaaccttcgtggacaggggatggggaaacggttgcggcctgttcggaaagggttccctgatcacctgcgctaagttcaagtgcgtcactaagctggagggcaagatcgtgcagtacgaaaacctgaagtactctgtcatcgtgactgtccacaccggagaccagcaccaggtcggtaacgagtcaaccgaacacggcactaccgctactatcacccctcaggccccaactaccgagatccagctgaccgactacggtgctctgacactcgactgcagcccccgcactggactggacttcaacgagatggtcctgctgactatgaaggaaaagtcctggctggtgcacaagcagtggttcctggacctgccactgccttggactagcggcgcctccaccagccaggagacttggaaccgtcaggacctgctggtgactttcaagaccgcccacgctaagaagcaggaggtggtcgtgctgggctctcaggaaggagccatgcacactgctctgaccggtgccactgaaatccagacctcaggtactaccactatcttcgctggccacctgaagtgccgcctgaagatggacaagctgaccctgaagggaatgtcttacgtcatgtgcactggctcattcaagctggagaaggaagtggctgagacccagcacggcactgtgctggtccagatcaagtacgaaggaaccgacgccccttgcaagatccccttcagcacccaggacgagaagggcgtcactcagaacggaaggctgatcaccgctaaccccatcgtcactgacaaggaaaagccagtgaacatcgaggccgaacctcccttcggagagtcttacatcgtgatcggcgctggagaaaaggccctgaagctgtcatggttcaagaagggttcttcaatcggcaagatgttcgaggccaccgctagaggtgctcgccgtatggccatcctgggagacaccgcttgggacttcggttccatcggcggagtgttcactagcgtcggcaagctggtgcaccagatcttcggaaccgcctacggtgtcctgttctctggcgtgtcatggaccatgaagatcggtatcggcgtgctgctgacttggctgggactgaactctcgttcaacctccctgagcatgacttgcatcgctgtgggcctggtcaccctgtacctgggagtgatggtccaggcc

SEQ ID NO: 14 - protein, 804 aa, synthetic construct
PRITEAEPDDVDCWCNATDTWVTYGTCSQTGEHRRDKRSVALAPHVGLGLETRTETWMSSEGAWKQIQRVETWALRHPGFTVIALFLAHAIGTSITQKGIIFILLMLVTPSMAKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSMRCVGIGSRDFVEGLSGATWVDVVLEHGSCVTTMAKDKPTLDIELLKTEVTNPAILRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNESTEHGTTATITPQAPTTEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQA

SEQ ID NO: 15 - protein, 691 aa, synthetic construct
KPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSMRCVGIGSRDFVEGLSGATWVDVVLEHGSCVTTMAKDKPTLDIELLKTEVTNPAILRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNESTEHGTTATITPQAPTTEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQA

SEQ ID NO: 16 - DNA, 4012 bp, synthetic construct
  source 1..4012: mol_type = other DNA
gagtcgtgttcataccgcatatgccaccatgggtggcaacgagggttccatcatgtggctggcttccctggctgtggtcatcgcttgcgccggtgctatgagtcttgccatcccagttatgtgcctgttggcaaacaccacgttcccctgctcccagcccccttgcacgccctgctgctacgaaaaggaaccggaggaaaccctacgcatgcttgaggacaacgtcatgagacctgggtactatcagctgctacaagcatccttaacatgttctccccaccgccagcgacgcagcacccgaaagccaaccgaaatcaacgaggacctgaacatcgctgccgtggcttccaacttcgccaccactgacctggaaaccgacctgttcactaactgggagaccatgaacgtcatcagcaccgacactgaacaggtgaacaccgacgctgaccgtggaaagctgcctggcaagaagctgcccccagacgtcctgagagagctggaagctaacgctaggagagctggatgcactaggggatgcctgatctgcctgtcccacatcaagtgcacccctaagatgaagaagttcatccccggaaggtgccacacttacgagggtgaaaaggagagcgctcagggcggaatcggcgaagccatcgtggacatccctgagatccccggattcaaggacaaggagcccctggaccagttcatcgctcaggtcgacctgtgcgccgactgcaccactggttgcctgaagggcctggctaacgtgcagtgctctgacctgctgaagaagtggctgccacagcgctgcaccactttcgcttcaaagatccagggtcgtgtggacaagatcaagggactggctggagacaggggcggtggctcagactctagaaacttcaatgtctataaagccacaagaccatacttagctcactgtcccgactgtggagaagggcactcgtgccatagtcccgtagcactagaacgcatcagaaatgaagcgacagacgggacgctgaaaatccaggtctccttgcaaatcggaataaagacggatgacagccacgattggaccaagctgcgttatatggacaaccacatgccagcagacgcagagagggcggggctatttgtaagaacatcagcaccgtgtacgattactggaacaatgggacacttcatcctggcccgatgtccaaaaggggaaactctgacggtgggattcactgacagtaggaagattagtcactcatgtacgcacccatttcaccacgaccctcctgtgataggtcgggaaaaattccattcccgaccgcagcacggtaaagagctaccttgcagcacgtacgtgcagagcaccgccgcaactaccgaggagatagaggtacacatgcccccagacacccctgatcgcacattaatgtcacaacagtccggcaacgtaaagatcacagtcaatggccagacggtgcggtacaagtgtaattgcggtggctcaggtggcggatctggtggcggatcaaagggcatgtcctacgctatgtgcaccggaaagttcaagctggagaaggaagtggccgagacccagcacggaactatcctgatcaaggtcaagtacgaaggtgacggcgctccatgcaagatccctttcgagatccaggacgtggaaaagaagcacgtcaacggccgcctgatcaccgctaaccccatcgtgactgacaaggagtcacctgtcaacatcgaggccgaacctcccttcggtgactcctacatcgtgatcggagtcggtgacaaggccctgaagctgaactggttcaagaagggatccagcggtggcggaagcggtggcggatctaatgaaggactaacaactacagacaaagtgattaataactgcaaggttgatcaatgtcatgccgcggtcaccaatcacaaaaagtggcagtataactcccctctggtcccgcgtaatgctgaacttggggaccgaaaaggaaaaattcacatcccgtttccgctggcaaatgtaacatgcagggtgcctaaagcaaggaaccccaccgtgacgtacgggaaaaaccaagtcatcatgctactgtatcctgaccacccaacactcctgtcctaccggaatatgggagaagaaccaaactatcaagaagagtgggtgatgcataagaaggaagtcgtgctaaccgtgccgactgaagggctcgaggtcacgtggggcaacaacgagccgtataagtattggccgcagttatctacaaacggtacagcccatggccacccgcatgagataattctgtattattatgagctgtaccccactatgactgtagtagttgtgtcagtggccacgttcatactcctgtcgatggtgggtatggcagcggggatgtgcatgtgtgcacgacgcagatgcatcacaccgtatgaactgacaccaggagctaccgtccctttcctgcttagcctaatatgctgcatcagaacagctaaagcggccacataccaagaggctgcgatatacctgtggaacgagcagcaacctttgttttggctacaagcccttattccgctggcagccctgattgttctatgcaactgtctgagactcttaccatgctgctgtaaaacgttggcttttttagccgtaatgagcgtcggtgcccacactgtgagcgcgtacgaacacgtaacagtgatcccgaacacggtgggagtaccgtataagactctagtcaatagacctggctacagccccatggtattggagatggaactactgtcagtcactttggagccaacactatcgcttgattacatcacgtgcgagtacaaaaccgtcatcccgtctccgtacgtgaagtgctgcggtacagcagagtgcaaggacaaaaacctacctgactacagctgtaaggtcttcaccggcgtctacccatttatgtggggcggcgcctactgcttctgcgacgctgaaaacacgcagttgagcgaagcacacgtggagaagtccgaatcatgcaaaacagaatttgcatcagcatacagggctcataccgcatctgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgtgggtacggtacacgtgccatactctcaggcaccatctggctttaagtattggctaaaagaacgcggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatctccgctacggcgatgtcatgggtgcagaagatcacgggaggtgtgggactggttgttgctgttgccgcactgattctaatcgtggtgctatgcgtgtcgttcagcaggcac

SEQ ID NO: 17 - protein, 1328 aa, synthetic construct
MGGNEGSIMWLASLAVVIACAGAMSLAIPVMCLLANTTFPCSQPPCTPCCYEKEPEETLRMLEDNVMRPGYYQLLQASLTCSPHRQRRSTRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSDSRNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSGGGSGGGSKGMSYAMCTGKFKLEKEVAETQHGTILIKVKYEGDGAPCKIPFEIQDVEKKHVNGRLITANPIVTDKESPVNIEAEPPFGDSYIVIGVGDKALKLNWFKKGSSGGGSGGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKAATYQEAAIYLWNEQQPLFWLQALIPLAALIVLCNCLRLLPCCCKTLAFLAVMSVGAHTVSAYEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAVGTVHVPYSQAPSGFKYWLKERGASLQHTAPFGCQIATNPVRAVNCAVGNMPISIDIPEAAFTRVVDAPSLTDMSCEVPACTHSSDFGGVAIIKYAASKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSRH

SEQ ID NO: 18 - protein, 740 aa, synthetic construct
STRKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDRGGGSDSRNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSGGGSGGGSKGMSYAMCTGKFKLEKEVAETQHGTILIKVKYEGDGAPCKIPFEIQDVEKKHVNGRLITANPIVTDKESPVNIEAEPPFGDSYIVIGVGDKALKLNWFKKGSSGGGSGGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA

SEQ ID NO: 19 - protein, 439 aa, synthetic construct; identical to SEQ ID NO: 4